(19) United States
(12) Patent Application Publication
(10) Pub. No.: US 2023/0018177 A1
(43) Pub. Date: Jan. 19, 2023

(54) BLOOD FILTRATION SYSTEM AND PLASMA VOLUME MONITORING

(71) Applicant: Nuwellis, Inc., Eden Prairie, MN (US)

(72) Inventors: David Lerner, St. Paul, MN (US); Franz Willems Ulrich, Minneapolis, MN (US); David J. Haskvitz, Maple Grove, MN (US)

(21) Appl. No.: 17/757,196

(22) PCT Filed: Dec. 31, 2020
(86) PCT No.: PCT/US2020/067730

§ 371 Date: Jun. 10, 2022

Related U.S. Application Data
(60) Provisional application No. 62/955,840, filed on Dec. 31, 2019.

(51) Int. Cl. A61M 1/34; A61M 1/36

(52) U.S. Cl. CPC A61M 1/3496; A61M 1/341; A61M 1/3609; A61M 2202/0415; A61M 2205/3306; A61M 2205/3334; A61M 2230/63

(57) ABSTRACT

A blood filtration system may include blood circuit configured to transmit a fluid within one or more lumens. The system may include an optical sensor configured to couple with the blood circuit. The optical sensor may measure one or more optical characteristics of the fluid in the blood circuit. The one or more optical characteristics may include a first optical characteristic corresponding to a concentration of an imaging substance in the fluid within the blood circuit. The system may include a controller in communication with the optical sensor. The controller may include a sampling module configured to record the one or more optical characteristics. The controller may include a physiological characteristic identification module configured to determine a plasma volume of the patient with the recorded optical characteristics of the imaging substance.

### CLAIM OF PRIORITY

This patent application claims the benefit of priority of Lerner et al., U.S. Provisional Patent Application Ser. No. 62/955,840, entitled “PLASMA AND BLOOD VOLUME MEASUREMENT DURING THERAPY,” filed on Dec. 31, 2019 (Attorney Docket No. 4567.030PRV), which is hereby incorporated by reference herein in its entirety.

### TECHNICAL FIELD

This document pertains generally, but not by way of limitation, to a blood filtration system.

### BACKGROUND

In an approach, plasma volume is measured by injecting a tracer or an indicator and manually drawing a blood sample from a patient, for example by withdrawing the blood into a vial. The blood sample may be analyzed to determine the plasma volume of the patient through detection of the tracer or indicator. In some approaches, a plurality of blood samples may be withdrawn from a patient and used to determine the plasma volume of the patient. For instance, the blood samples may be manually drawn by a healthcare provider (e.g., a technician, nurse, doctor, or the like) at a specific time frequency (e.g., one sample every 30 seconds) to determine the plasma volume of the patient through detection of the tracer or indicator in each of the drawn samples.

### SUMMARY

The hematocrit value in a patient is a ratio of the red blood cell volume to the total volume of blood in a patient, where the total volume of blood includes the total body red blood cell volume (in contrast to the volume of a single, individual, red blood cell) and the volume of plasma (including, but not limited to water, proteins, and electrolytes) in the blood of the patient. In some examples, a patient experiencing congestive heart failure may have excess plasma volume, and accordingly a reduced hematocrit value. For instance, the patient may have excess plasma water that correspondingly increases the plasma volume of the patient (and lowers the hematocrit value of the patient). A blood filtration system may reduce the amount of plasma constituents (e.g., water and/or electrolytes) in the blood of the patient, and accordingly increase the hematocrit value of the patient by decreasing the volume of plasma relative to red blood cell volume.

The present inventors have recognized, among other things, that a problem to be solved may include determining a plasma volume of a patient without repeated blood draws conducted by a healthcare provided. Additionally, present inventors have recognized, among other things, that a problem to be solved may include monitoring and guiding (e.g., controlling, or the like) blood filtration therapy of a patient, for instance to determine when to end blood filtration therapy for the patient. For example, a known quantity (e.g., 5 milliliters, 10 milliliters, 5 milligrams, 10 milligrams, or the like) of a imaging substance, such as indocyanine green (“ICG”), may be infused into a patient. A plurality of blood samples may be withdrawn by a healthcare practitioner at a specified time frequency after the infusion of the imaging substance. The blood samples may be analyzed, for instance to determine a concentration of the imaging substance in each blood sample. The concentration of the imaging substance in the blood sample may correspond to the plasma volume of the patient, for instance because the plasma volume may be determined using the known quantity of the infused imaging substance and the concentration of the imaging substance in each blood sample.

In an approach, it may be challenging for a healthcare provider to determine the plasma volume of a patient. For example, accuracy of the plasma volume determination may be affected by a rate that the imaging substance is infused. In another example, the imaging substance may be cleared by physiological processes. For instance, the liver may clear the imaging substance by removing the imaging substance from the blood stream of the patient. The clearance of the imaging substance affects the concentration of the imaging substance in the blood stream of the patient. Accordingly, clearance of the imaging substance may decrease the accuracy of plasma volume determinations where blood is manually withdrawn from a patient because the plasma volume appears artificially low as the imaging substance is cleared. Further, albumin concentration in blood may affect plasma volume determinations for the blood, for instance by producing an oncotic pressure differential that changes the plasma volume of a patient.

The present subject matter may help provide a solution to these problems, such as by a blood filtration system that automatically monitors plasma volume and corresponding hematocrit values and filters blood based on the monitoring. The blood filtration system may include an extracorporeal blood circuit. The blood circuit may transmit a fluid within one or more lumens, for instance a withdrawal lumen and an infusion lumen. The lumens are configured for communication (e.g., fluidic communication, or the like) with vasculature of a patient. For instance, the blood circuit may include a catheter, and the catheter may be inserted into the vasculature of the patient. The lumens are in communication with the catheter, and accordingly in communication with the vasculature (with the catheter inserted into the vasculature). In an example, the blood filtration system withdraws blood through the catheter and into the withdrawal lumen. The withdrawal lumen is in communication with a filter, and the filter optionally removes one or more plasma constituents of the blood. The (optionally filtered) blood may be discharged from the filter and into the infusion lumen. The infusion lumen may supply the (optionally filtered) blood to the catheter, and the catheter may infuse the (optionally filtered) blood into the vasculature of the patient. Accordingly, the blood filtration system facilitates removal of one or more plasma constituents from blood of a patient.

As described herein, the extracorporeal blood circuit may include a filter. The blood filtration system may include a filtration pump, and the filtration pump may extract a filtrate fluid from the filter. The filter may remove one or more plasma constituents, such as water, from blood flowing through the filter. The filtrate fluid may include the one or more plasma constituents removed from the blood, and the filtration pump may extract the filtrate fluid from the filter. For instance, the filtration pump may pump the filtrate fluid to a container (e.g., a bag, vessel, or the like) for collection. The filter may discharge the filtered blood, for instance by supplying the filtered blood to the infusion lumen for infusion into the patient. Accordingly, the blood filtration system may remove the one or more plasma constituents from the blood of the patient.

In another example, the blood filtration system may include an optical sensor, and the optical sensor may couple with the extracorporeal blood circuit.

For instance, the extracorporeal blood circuit may include a cuvette in communication with the one or more lumens. Blood (or other fluid) may flow through the lumens, including the cuvette. The optical sensor may couple with the cuvette, for instance to measure one or more optical characteristics of the blood (or other fluid) in the blood circuit (including the cuvette).

Optical characteristics of the blood include one or more of a wavelength, bandwidth, intensity, frequency, duration, or the like of light transmitted through the blood and received by the optical sensor. Optical characteristics of the blood may vary in correspondence with concentrations of substances within the blood, for instance red blood cells or an imaging substance (e.g., indocyanine green, or the like). In an example, optical characteristics of blood may vary in correspondence with a concentration of imaging substance in blood infused in a patient.

The optical sensor may include a photoemitter that emits light at a specified wavelength (e.g., 810 nm, 800 nm to 820 nm, or the like) and a specified intensity. As described herein, the optical sensor facilitates ongoing monitoring of the blood including blood characteristics such as, but not limited to, plasma volume, hematocrit value or the like. The specified wavelength of light is absorbed by the imaging substance, and the optical sensor receives the light reduced by the imaging substance absorption of the light emitted by the optical sensor. Intensity of the light received by the photoreceiver may vary in correspondence with the concentration of imaging substance in the blood. In an example, the optical sensor may include a photoreceiver, and the photoreceiver may measure one or more of wavelength, bandwidth, intensity, frequency, duration, or the like of light received (after transmission through the blood filtration system). For instance, the photoreceiver may measure the intensity of light at the specified wavelength. The measured intensity of light at the specified wavelength may correspond to the concentration of the imaging substance (e.g., ICG, or the like) within the blood of the patient. For instance, the photoreceiver may measure an increase in intensity of light at the specified wavelength when the concentration of the imaging substance correspondingly decreases in the blood of the patient. Optionally, the system supplements the imaging substance in the blood to maintain a specified concentration of the imaging substance. The optical sensor may measure one or more optical characteristics of fluid in the extracorporeal blood circuit, for instance to facilitate determining the plasma volume of the patient according to the measured one or more optical characteristics of the fluid (e.g., through detection of the imaging substance). Accordingly, in an example, the blood filtration system facilitates determining the plasma volume (and optionally maintains and controls infusion of the imaging substance, such as ICG) while a healthcare provider refrains from collecting a manual blood sample (e.g., by withdrawing blood into a vial) to determine the plasma volume. In another example, the fluid having optical characteristics (e.g., ICG, or the like) measured by the optical sensor is infused into the patient.

In an example, the optical sensor 130 may measure one or more optical characteristics of fluid in the blood circuit 120. For instance, the photoemitter 600 may generate light at one or more wavelengths to help determine concentration of an imaging substance or to help determine hematocrit of a patient (e.g., based on the determined concentration of the imaging substance). Accordingly, the optical sensor 130 may help determine a plurality of physiological characteristics of a patient, such as plasma volume, hematocrit, red blood cell volume, or the like.

In an example, the blood filtration system may include a controller having processing circuitry. The controller may be in communication with the optical sensor. For example, the controller may include a sensor interface module that receives the measured one or more optical characteristics from the optical sensor. The controller may include a sampling module, and the sampling module facilitates recording optical characteristics, for instance measured optical characteristics received by the sensor interface. For instance, the sampling module may record the measured optical characteristics with respect to time. In another example, the sampling module records a first measured optical characteristic a first intensity at a specified wavelength, or the like) at a first time and a second measured optical characteristic (e.g., a second intensity at the specified wavelength, or the like) at a second time. The optical characteristics may include linear combinations of multiple wavelengths of light. As described herein, the blood filtration system may include the controller. The controller may include a physiological characteristic identification module, and the physiological characteristic identification module may help determine the plasma volume of the patient with the recorded optical characteristics. For instance, a specified volume of the imaging substance is infused into the patient, and the physiological characteristic identification module determines the plasma volume. In one example, the plasma volume is determined by dividing (e.g., with a mathematical operation, or the like) the infused volume of the imaging substance that is infused into a patient by a concentration of the imaging substance detected in analyzed blood (e.g., with the optical sensor).

In an example, the physiological characteristic identification module may determine the concentration of the imaging substance in the fluid within the blood circuit based on the optical characteristics of the fluid measured by the optical sensor. For instance, the physiological characteristic identification module may correlate the imaging substance with its characteristic absorption wavelength. In another example, the physiological characteristic identification module may correlate the imaging substance with its degree of ultimate attenuation. In yet another example, the concentration of the imaging substance is determined according to the Beer-Lambert law, or the like.

As discussed herein, the blood filtration system facilitates determining the plasma volume of the patient. For instance, the blood filtration system may guide therapy conducted by the blood filtration system according to the determined plasma volume of the patient. In an example, the physiological characteristic identification module repeatedly determines (e.g., in an ongoing manner) the plasma volume of the patient, for instance while the blood filtration system removes one or more plasma constituents from the blood of the patient (e.g., with a filter). For example, the blood filtration system may modulate one or more pumps to adjust the speed of the pumps (and corresponding flow rate of the blood and filtration rate) based on the determined plasma volume of the patient. In an example, the controller includes a pump module, and the pump module may adjust a speed, flow rate, output or the like of the filtration pump based on the determined plasma volume of the patient. In another example, the pump module may modulate the filtration pump and may adjust a speed of the filtration pump when the plasma volume of the patient exceeds a specified threshold (e.g., euvolemia based on patient-specific needs, or the like). In another example, the pump module may stop the filtration pump when the specified threshold is met (e.g., achieved, exceeded, fallen below or the like). Accordingly, the blood filtration system in one example provides an automated system that provides ongoing monitoring of the plasma volume of a patient and at the same time controls therapy conducted with the blood filtration system according to the ongoing monitoring. The ongoing monitoring and control of therapy provide a real time control system in contrast to disjointed monitoring and follow on therapy conducted with blood drawing and subsequent filtration based on the assessment of (potentially outdated) previous blood draws.

The blood filtration system may enhance one or more of accuracy or precision of plasma volume determinations. As described herein, the optical sensor may measure optical characteristics of fluid in the actual blood circuit in communication with the patient. For instance, the optical sensor may measure the optical characteristics of the fluid without removing the fluid from the blood circuit and the vasculature of the patient in communication with the system. Accordingly, the blood filtration system may measure optical characteristics of fluid in the blood circuit without taking a sample of the fluid (e.g., by extracting the fluid from the blood circuit). Thus, the resolution (e.g., number of measurements per unit of time, or the like) of optical characteristic measurements may be enhanced, for instance because the optical sensor may measure the optical characteristics of the fluid in the blood circuit at a consistent rate, over a specified time period or the like in comparison to blood samples obtained manually by blood draws with a healthcare provider. For instance, the optical sensor may conduct ongoing or continuous measurements (e.g., 5 measurements a minute, 5 measurements a second, 100 measurements a second, or the like) of the optical characteristics of the fluid, such as during therapy to remove one or more plasma constituents from the blood. Accordingly, the enhanced resolution of measurements provided by the optical sensor may correspondingly enhance the accuracy (or precision) of the blood filtration system determining the plasma volume of the patient. For example, the accuracy (or precision) of plasma volume determinations by the blood filtration system are enhanced because the physiological characteristic identification module determines the concentration of the imaging substance in the fluid within the blood circuit based on the (enhanced resolution) measurements of the optical characteristics of the fluid. As discussed herein, the enhanced resolution facilitates real time control of blood filtration, infusion of imaging substances or the like.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components.

The drawings illustrate generally, by way of example, but not by way of limitation, various embodiments discussed in the present document.

FIG. 1 illustrates a schematic view of an example of portions of a blood filtration system, according to an embodiment of the present subject matter.

FIG. 2 illustrates a perspective view of an example of a cuvette, according to an embodiment of the present subject matter.

FIG. 3 illustrates a cross-sectional view of the cuvette of FIG. 2 at the line 3-3, according to an example of the present subject matter.

FIG. 4 illustrates a perspective view of another example of the cuvette, according to an embodiment of the present subject matter.

FIG. 5 illustrates a side view of the cuvette of FIG. 4 and an optical sensor, according to an embodiment of the present subject matter.

FIG. 6 illustrates another side view of the cuvette of FIG. 4 and the optical sensor, according to an embodiment of the present subject matter.

FIG. 7 illustrates a schematic view of another example of the blood filtration system, according to an embodiment of the present subject matter.

FIG. 8 illustrates a graphical representation of a plot including a concentration of an imaging substance with respect to time, according to an embodiment of the present subject matter.

FIG. 9 illustrates a diagram of an example of a method for removing one or more plasma constituents from fluid in vasculature of a patient, according to an embodiment of the present subject matter.

FIG. 10 illustrates a schematic view of another example of the filtration system, according to an embodiment of the present subject matter.

FIG. 11 illustrates one example of a method 1100 for determining a patient-specific imaging substance dosage, according to an embodiment of the present subject matter,

FIG. 12 illustrates another cross-sectional view of the cuvette of FIG. 2 at the line 3-3, according to an example of the present subject matter.

FIG. 13 illustrates a block diagram of an example machine upon which any one or more of the techniques discussed herein may perform.

### DETAILED DESCRIPTION

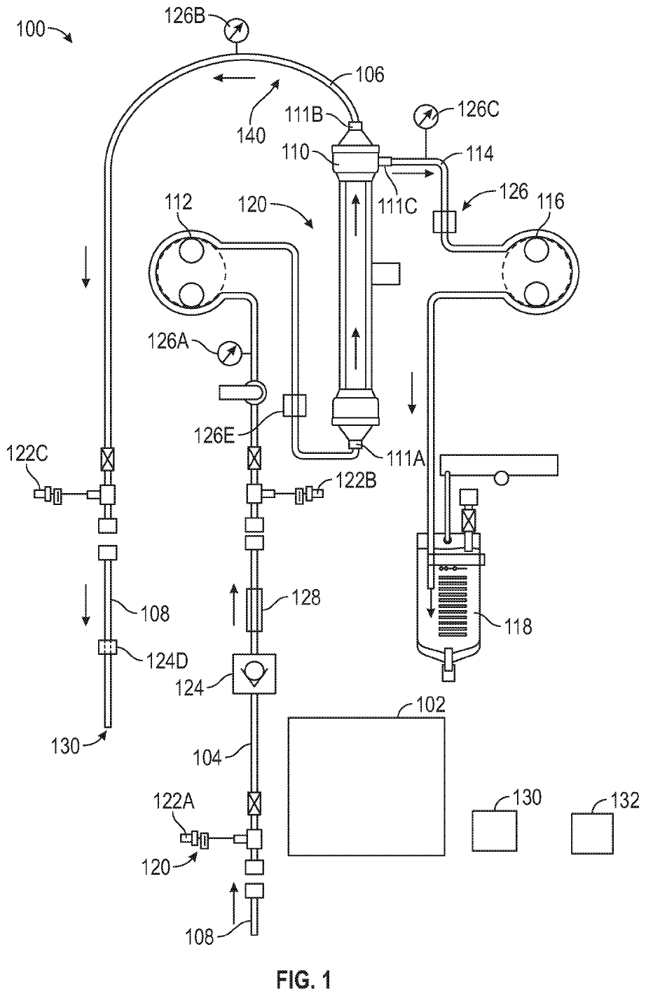

FIG. 1 illustrates a schematic view of an example of portions of a blood filtration system 100, according to an embodiment of the present subject matter. The blood filtration system 100 may reduce one or more plasma constituents (e.g., water, proteins, electrolytes, or the like) in blood of a patient. The blood filtration system 100 may facilitate one or more blood filtration operations, including (but not limited to): extracorporeal ultrafiltration, continuing renal replacement therapy (“CRRT”), slow continuous ultrafiltration (“SCUF”), continuous veno-venous hemofiltration (“CVVH”), continuous veno-venous hemofiltration (“CVVHD”), dialysis, continuous veno-venous hemofiltration including dialysis and filtration (“CVVHDF”), sustained low efficiency dialysis (“SLED”), extracorporeal membrane oxygenation (“ECMO”) therapy, modified ultrafiltration, and peripheral plasmapheresis, peripheral hemofiltration.

The blood filtration system 100 may include a controller 102. The controller 102 may include processing circuitry, for instance an integrated circuit or the like. As described herein, the controller 102 may be configured to control one or more components, functions, features, operations, or the like of the blood filtration system 100.

The blood filtration system 100 may include a withdrawal line 104 and may include an infusion line 106. The lines 104, 106 may be configured to couple with a catheter 108, and the lines 104, 106 may transmit blood within the blood filtration system 100. In an example, the catheter 108 may be inserted into a blood stream of the patient, for instance the catheter 108 may be inserted into a basilic vein, cephalic vein, brachial vein, the axillary vein, the subclavian vein, the brachiocephalic vein, or the like. Blood may flow into the catheter 108, into the withdrawal line 104, through other components of the system 100, through the infusion line 106, into the catheter 108, and back into the blood stream of the patient. The line 104 may be separate from the line 106. The lines 104, 106 may be in communication with the catheter 108. For example, the catheter 108 may include one or more lumens, for example a withdrawal lumen in communication with the line 104 and an infusion lumen in communication with the line 106.

The lines 104, 106 may be configured to couple with a filter 110, for instance the lines 104, 106 may include one or more fittings that facilitate coupling the lines 104, 106 with the filter 110. In an example, the withdrawal line 104 may couple with a filter inlet port 111A, and the infusion line 106 may couple with a filter outlet port 111B. The filter 110 may be configured to reduce an amount of one or more plasma constituents water, electrolytes, or the like) in blood flowing through the filter 110 and provide a filtrate fluid including the one or more plasma constituents. As described herein, blood may flow through the lines 104, 106 to and from the catheter 108. The lines 104, 106 may be coupled with the filter and blood may flow from the withdrawal line 104, through the filter 110, and into the infusion line 106.

The blood filtration system 100 may include a blood pump 112, and the blood pump 112 may pump (e.g., convey, drive, push, or the like) blood through the blood filtration system 100. In an example, the blood pump 112 may be a peristaltic pump, and the blood pump 112 may engage with the withdrawal line 104 to pump blood through the withdrawal line 104 and into the filter 110. The controller 102 may be configured to operate the blood pump 112 to vary a speed of the blood pump 112 and accordingly vary the flow rate of blood through the blood filtration system 100 (e.g., the withdrawal line 104, the filter 110, the infusion line 106, or the like).

Referring again to FIG. 1, the blood filtration system 100 may include a filtration line 114 and a filtration pump 116. The filtration line 114 may be configured to couple with the filter 110 (e.g., with a fitting), for instance the filtration line 114 may couple with a filtrate fluid port 111C. The filter 110 may be configured to transmit the filtrate fluid (including one or more plasma constituents) extracted by the filter 110 to the filtrate fluid port 111C.

The filtration pump 116 may pump extracted filtrate fluid from the filter 110, and into a filtrate fluid reservoir 118 (e.g., a bag, container, bladder, or the like). In some examples, the filtration pump 116 may be a peristaltic pump that engages with the filtration line 114 to pump the filtrate fluid through the filtrate fluid line 114. The controller 102 may be configured to vary a speed of the filtration pump 116 and accordingly vary the flow rate of filtrate fluid through the blood filtration system 100 (e.g., the filtration line 114).

The system 100 may include a blood circuit 120, and the blood circuit 120 may include one or more components of the system 100, such as may provide a conduit for blood flow. For example, the blood circuit 120 may include (but is not limited to) the withdrawal line 104, the infusion line 106, the catheter 108, the filter 110, the filtration line 114, the filtrate fluid reservoir 118. The blood circuit 120 may include components of the system 100 that are in communication with a biological fluid of the patient.

In some examples, the blood filtration system 100 may include one or more access ports 122, for instance a first access port 122A, a second access port 122B, and a third access port 122C. The access ports 122 may facilitate the extraction of blood from the blood filtration system 100, or injection of substances (e.g., imaging substance, a blood thinner, for instance heparin or the like) into the blood within the blood filtration system 100. In an example, the access ports 122A, 122B may be in communication with the withdrawal line 104, and the access port 122C may be in communication with the infusion line 106.

A valve 124 (e.g., a mechanical check valve, or electronically controlled valve) may be positioned between the access ports 122A, 122B, and the valve 124 may be configured to allow blood to flow unidirectionally within the withdrawal line 104 (e.g., flowing from the catheter 108 to the filter 110). In this example, a substance may be injected into the withdrawal line 104 at the access port 122B, and blood may be withdrawn from the access port 122A. Because the valve 124 facilitates unidirectional flow within the withdrawal line 104, the blood including the substance will not be withdrawn from the access port 122A, for instance because the access port 122A is upstream of the access port 120B).

In an example, heparin may be infused into the access port 122B and blood is drawn from the access port 122A to measure blood clotting time parameters of a patient. Because the blood is drawn from the access port 122A, the withdrawn blood does not include heparin, and in an example, a blood clotting time parameter determination is not affected by the heparin injection at the access port 122B. Accordingly, the performance of blood filtration system 100 is thereby improved.

As shown in FIG. 1, the blood filtration system 100 may include one or more sensors 126 (e.g., transducer, accelerometer, or the like), for instance a first sensor 126A, a second sensor 126B, and a third sensor 126C. The first sensor 126A may measure (e.g., obtain, provide, quantify, evaluate, or the like) the pressure within the withdrawal line 104, the second sensor 126B may measure the pressure within the infusion line 106, and the third sensor 126C may measure the pressure within the filtration line 114. The sensors 126 may include a fourth sensor 126D (e.g., a position sensor, or the like) and a fifth sensor 126E (e.g., blood flow rate, or the like), and the sensor 126E may determine the blood flow rate through the system 100 (e.g., a component of the blood circuit 120, for example the withdrawal line 104).

In an example, the blood filtration system includes a cuvette 128 in communication with the blood circuit 120. For example, the cuvette 128 may be in communication with one or more of the withdrawal line 104 or the infusion line 106. In another example, fluid (e.g., blood including one or more plasma constituents, or the like) may be withdrawn from vasculature of a patient and flow through the withdrawal line 104, for instance through a withdrawal lumen of the withdrawal line 104. The cuvette 128 may be in communication with the withdrawal line 104, and fluid flowing through the withdrawal line 104 may flow through the cuvette 128. As described herein, the cuvette may include an optical window that facilitates measurement of optical characteristics of the fluid in the cuvette 128, In an example, optical characteristics of the blood include one or more of a wavelength, bandwidth, intensity, frequency, duration, or the like of light transmitted through the fluid and received by an optical sensor, Optical characteristics of the fluid may vary in correspondence with concentrations of substances within the blood, for instance a concentration of red blood cells or a concentration of an imaging substance (e.g., indocyanine green, or the like). In an example, optical characteristics of fluid may vary in correspondence with a concentration of indocyanine green infused in the blood stream of a patient.

Referring to FIG. 1, the blood filtration system 100 may include an optical sensor 130, and the optical sensor 130 may be included in the sensors 126. The optical sensor 130 may measure optical characteristics of fluid in the blood circuit 120 (e.g., fluid in the cuvette 128, or the like). The optical sensor 130 may couple with the blood circuit 120, for instance coupling with cuvette 128. The optical sensor 130 may transmit light through the cuvette 128 to measure the optical characteristics of fluid received in the cuvette 128.

FIG. 1 shows the blood filtration system 100 may include a hematocrit sensor 132, and the hematocrit sensor 132 may be included in the sensors 126. In some examples, the hematocrit sensor 132 is an optical hematocrit sensor. Optionally, the hematocrit sensor 132 may be included in the optical sensor 130, The hematocrit sensor 132 may be coupled with the blood circuit 120, for instance with one or more of the lines 104, 106 or the cuvette 128, The hematocrit sensor 132 may measure a hematocrit value (e.g., level, or the like) of the patient. In an example, the hematocrit sensor 132 may be located between the catheter 108 and the valve 124, for instance to monitor the hematocrit value of the patient prior to injection of a fluid (e.g., heparin or saline) into the blood (e.g., at the access port 122B). Accordingly, the hematocrit value determination may be improved with the system 100.

In an example, the controller 102 may be configured to control the speed of the blood pump 112 and set the flow rate of blood through the filter 110 at a first blood flow rate. Additionally, the controller 102 may be configured to control the speed of the blood pump 112 and set the flow rate of blood through the filter at a second blood flow rate. The first blood flow rate may be different than the second blood flow rate. The controller 102 may determine the hematocrit at the second blood flow rate. The controller 102 may control the speed of the blood pump 112 and set the flow rate of blood at the first blood flow rate after determining the hematocrit value, for example after determining the hematocrit value at the second blood flow rate.

The controller 102 may control the speed of the blood pump 112 to measure the hematocrit value because the hematocrit value may vary according to the speed of the blood pump 112 (or flow rate of fluid through the blood circuit, such as the cuvette 128) and controlling the speed of the blood pump may improve the accuracy of the hematocrit value determination. For instance, the hematocrit value determination by the hematocrit sensor 132 may be affected by the flow rate of blood through the filter. In this example, the speed of the blood pump 112 can vary, and the determined hematocrit value of the patient may vary according to the speed of the blood pump 112. Accordingly, measuring the hematocrit value with the blood pump 112 at a consistent speed may improve the accuracy of the hematocrit value determination. Accordingly, varying the speed of the blood pump 112 may account for a source of error in determining the hematocrit value and the performance of the blood filtration system 100 is thereby improved.

In another example, the sampling module 702 records the hematocrit value at a plurality of blood flow rates. For instance, the pump module 706 may modulate the blood pump 112 to pump blood through the blood circuit 120 at a first blood flow rate. The identification module 702 may determine a first hematocrit value at the first blood flow rate. The pump module 706 may modulate the blood pump 112 to pump blood through the blood circuit 120 at a second blood flow rate. The identification module 702 may determine a second hematocrit value at the first blood flow rate. In one example, the calibration module 707 may determine an error value between the first hematocrit value and the second hematocrit value to correct for potential measurement variations due to changes in blood flow rate. In another example, the controller 102, such as the calibration module 707, may generate a look up table based on recorded hematocrit values at the plurality of blood flow rates. For example, the look up table may facilitate correction of potential errors in hematocrit value determinations due to variations in blood flow rate by applying a hematocrit correction to the determined hematocrit values. The hematocrit correction may correspond to the error value between the first hematocrit value and the second hematocrit value. Accordingly, the system 100 may compensate for potential errors in hematocrit value determinations due to variations in blood flow rate through the blood circuit 120.

Create a HCT versus blood flow rate curve and calibrate results based on this. Now, the same may be true for the plasma volume measurement itself and the same techniques may eliminate this variance.

As described herein, the blood filtration system 100 may determine a hematocrit value of a patient. The hematocrit value of the patient may be expressed as:

$$Hematocrit Value = \frac{Red Blood Cell Volume}{Total Blood Volume}$$

In another example, the total blood volume is equal to the red blood cell volume plus the plasma volume. Accordingly, the hematocrit value of the patient may be expressed as:

$$Hematocrit Value = \frac{Red Blood Cell Volume}{Red Blood Cell Volume + Plasma Volume}$$

In yet another example, red blood cell volume of a patient may be expressed as:

$$Red Blood Cell Volume = \frac{Plasma Volume \times Hematocrit Value}{1 - Hematocrit Value}$$

Thus, total body red blood cell volume (in contrast to the volume of a single, individual, red blood cell) of a patient may be determined by dividing a first quantity by a second quantity. The first quantity may be equal to the plasma volume multiplied by the hematocrit value of the patient. The second quantity may be equal to 1 minus the hematocrit value of the patient (e.g., a number between 0 and 1). As described herein, the controller 102 (including identification module 704) may determine one or more of the plasma volume or the hematocrit value of the patient. Accordingly, the controller 102 (including identification module 704) may determine the red blood cell volume of a patient. In another example, the controller 102 repeatedly determines the red blood cell volume of a patient, for example based on measured optical characteristics of fluid in the blood circuit 120.

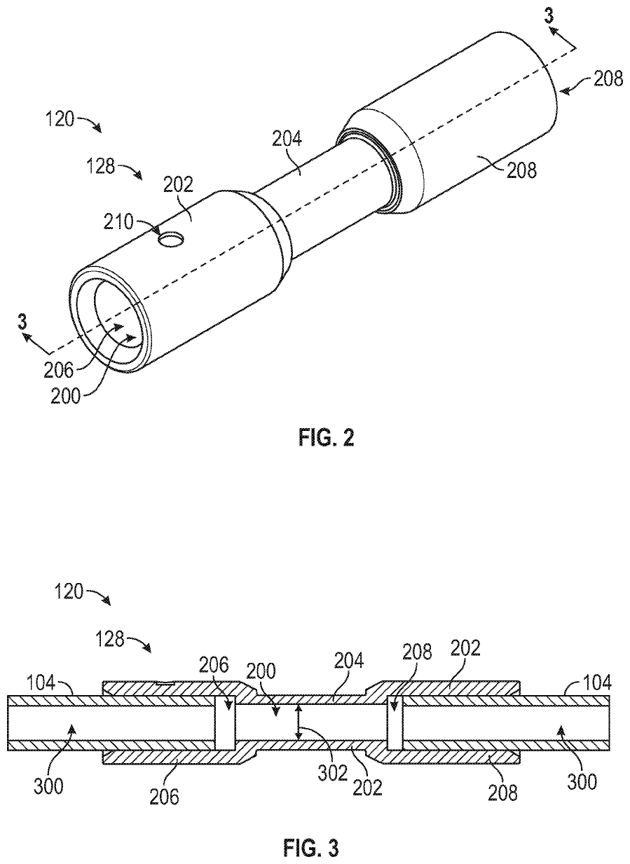

In an example, the blood filtration system 100 uses the red blood cell volume to determine a quantity of one or more plasma constituents to extract from the patient. In this example, the red blood cell volume may be input into the blood filtration system 100, for instance at the beginning of therapy. In another example, the controller 102 determines the red blood cell volume, for instance by determining one or more of the plasma volume or the hematocrit value of a patient based on measured optical characteristics of fluid in the blood circuit 120 (e.g., blood in the cuvette 128 shown in FIG. 2, or the like). In some examples, the controller 102 is optionally configured to set an extraction rate of filtrate fluid from the filter 110 using one or more of the determined red blood cell volume, the determined plasma volume, or the determined hematocrit value of the patient.

In another example, the blood filtration system 100 may determine a plasma refill rate of a patient. For instance, the plasma refill rate may include a rate that the plasma volume changes with respect to time, from within the body of the patient (in contrast to a change in plasma volume due to removal of water by the filter). For example, the plasma refill rate may include a rate that plasma water flows into intravascular space from interstitial spaces of the body. The controller 102 may determine the plasma refill rate, for instance by repeatedly determining the plasma volume of a patient and determining a change in the plasma volume of the patient.

The plasma refill rate of a patient may affect the plasma volume determination by the blood filtration system 100. For instance, the plasma refill rate may vary according to one or more factors, including (but not limited to) plasma oncotic pressure, plasma hydrostatic pressure, interstitial osmotic pressure, interstitial hydrostatic pressure, pressure derived from skin turgidity and elasticity, or the like. Accordingly, the plasma refill rate may vary from patient to patient or for one particular patient as therapy proceeds (e.g., the plasma refill rate may be dynamic during therapy for a patient). In an approach, if the extraction rate exceeds the plasma refill rate, the blood filtration system 100 may remove too much plasma fluid from the circulatory system too quickly (e.g., the extraction rate is too high) or in an absolute sense (e.g., total amount of plasma fluid removed during therapy exceeded a specified total). The removal of too much plasma fluid from the circulatory system may lead to hemoconcentration of the blood (excessive rise in hematocrit) with its attendant hyper-viscosity which may increase the likelihood of clotting in the filter. Further, in some approaches, if plasma volume is reduced to a low enough level, the patient may experience reduced blood pressure and tissue perfusion. Accordingly, the blood filtration system helps determine one or more of the plasma refill rate, plasma volume, red blood cell volume, or hematocrit. For instance, the blood filtration system 100 may guide therapy based on the plasma refill rate of the patient, for example by adjusting the extraction rate of filtrate fluid from the filter in proportion to the plasma refill rate.

In an example, the blood filtration system 100 may determine the plasma refill rate and use the plasma refill rate to determine when to stop therapy of the patient, for instance when the patient has reached euvolemia. The controller 102 may determine a quantity of filtrate fluid to extract from the patient. The controller 102 may determine how much plasma remains in the patient at a given point in time, for example to notify a healthcare provider for diagnostic purposes.

In another example, the blood filtration system 100 may guide therapy to provide a patient with an individualized extraction rate and an individualized target plasma volume. For example, the controller 102 may monitor a change in plasma volume of a patient relative to a total starting blood volume of the patient (e.g., a change in plasma volume divided by a starting plasma volume). In an approach, if plasma volume of a patient is above normal, the hematocrit value of the patient may be below a hematocrit threshold. For instance, if the hematocrit value of a patient is below the hematocrit threshold, the controller 102 may determine the patient's blood is hemo-diluted. In another example, as plasma fluid is removed (e.g., with the filter 110), the blood becomes may become hemo-concentrated (e.g., because the filter 110 may refrain from extracting red blood cells). Thus, the controller 102 may monitor changes in the hematocrit value of the patient, for instance to determine relative changes (e.g., on a percentage basis) in plasma volume of the patient. Accordingly, the blood filtration system measures optical characteristics of fluid in the blood circuit, for example to determine plasma volume of a patient (and changes of the plasma volume) during therapy with the blood filtration.

In another example, the blood filtration system 100 determining the red blood cell volume facilitates diagnoses of various types of anemia. For instance, the system 100 may determine the plasma volume and hematocrit value of a patient, and accordingly the identification module 704 may determine the red blood cell volume for the patient. The determined red blood cell volume of a patient may facilitate diagnosis of one or more of dilutional anemia or anemia. For instance, dilutional anemia may include an elevated plasma volume (e.g., in comparison to an expected value given patient demographics, or the like). Anemia may include a diminished red blood cell volume. Accordingly, by determining the plasma volume and red blood cell volume of a patient, the system 100 may help differentiate between dilutional anemia or anemia.

FIG. 2 illustrates a perspective view of an example of the cuvette 128, according to an embodiment of the present subject matter. As described herein, the cuvette 128 is included in the blood circuit 120, and the cuvette receives fluid, such as blood. In an example, the cuvette 128 may include a cuvette lumen 200. For instance, a cuvette wall 202 may surround the cuvette lumen 200. The cuvette wall 202 may include a translucent material, for example glass, plastic, or the like. The cuvette 128 includes an optical window 204 that facilitates measurement of optical characteristics of fluid in the cuvette lumen 200. For instance, the optical sensor 130 (shown in FIG. 1) couples with the cuvette 128, and the optical sensor 130 transmits light (e.g., light at a specified wavelength, intensity, or the like) through the optical window 204 to measure the optical characteristics of the fluid in the cuvette lumen 200. In another example, the optical window 204 is optimized to facilitate transmission of light through the cuvette lumen 200 (instead of around the cuvette lumen 200, such as by refracting light through the cuvette wall 202).

In an example, the cuvette 128 includes one or more of a first socket 206 or a second socket 208 (also shown in FIG. 3). For instance, the sockets 206, 208 are configured to receive a portion of the blood circuit 120, such as the withdrawal line 104 or the infusion line 106. In another example, the cuvette 128 is located in-line with the withdrawal line 104. Accordingly, the cuvette 128 may be in communication with the withdrawal line 104. For instance, fluid flowing in the withdrawal line 104 may flow through cuvette lumen 200. Thus, fluid may flow through the blood circuit 120, and the cuvette 128 may facilitate measurement of the optical characteristics of the fluid.

In an example, the cuvette 128 includes one or more coupling features 210. For instance, the coupling features 210 may include (but are not limited to) an indentation, ridge, key, keyway, groove, hole, protrusion, or the like) that facilitates alignment of the cuvette 128 relative to the optical sensor 130 (shown in FIG. 5). FIG. 2 shows the coupling features 210 including a hole 212, and the hole 212 optionally engages with a corresponding coupling feature (e.g., a pin, or the like) of the optical sensor 130, for instance to align the cuvette 128 relative to the optical sensor 130. In another example, the coupling features 210 facilitate alignment of the cuvette 128 relative to the optical sensor 130, for instance to align one or more of a photoemitter or a photoreceiver with the optical window 204.

FIG. 3 illustrates a cross-sectional view of the cuvette 128 of FIG. 2 at the line 3-3, according to an example of the present subject matter. As described herein, the cuvette 128 includes the sockets 206, 208 and the sockets 206, 208 may receive a portion of the blood circuit 120, for instance the withdrawal line 104. For instance, FIG. 3 shows the cuvette 128 located in-line with the withdrawal line 104. Accordingly, fluid (e.g., blood including one or more plasma constituents, or the like) may flow through a withdrawal lumen 300 and into the cuvette lumen 200. The optical window 204 facilitates measurement of optical characteristics of the fluid in the cuvette lumen 200. In an example, fluid flows from the cuvette lumen 200 back into the withdrawal lumen 300 and through the remainder of the blood circuit (e.g., through a filter and infused back into the patient).

As describe herein, the cuvette 128 includes the optical window 204, and the optical window 204 has a first characteristic 302, such as a length, radius, diameter, or the like. In an example, the first characteristic 302 corresponds to a diameter of the cuvette lumen 200. In another example, the first characteristic 302 corresponds to a distance that light travels through the cuvette lumen 20 (and the fluid optionally included therein).

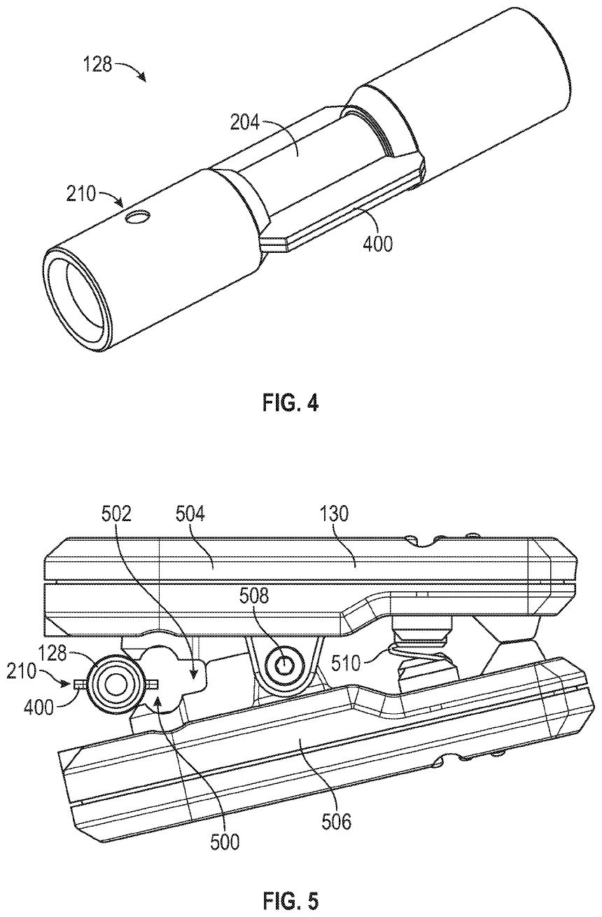

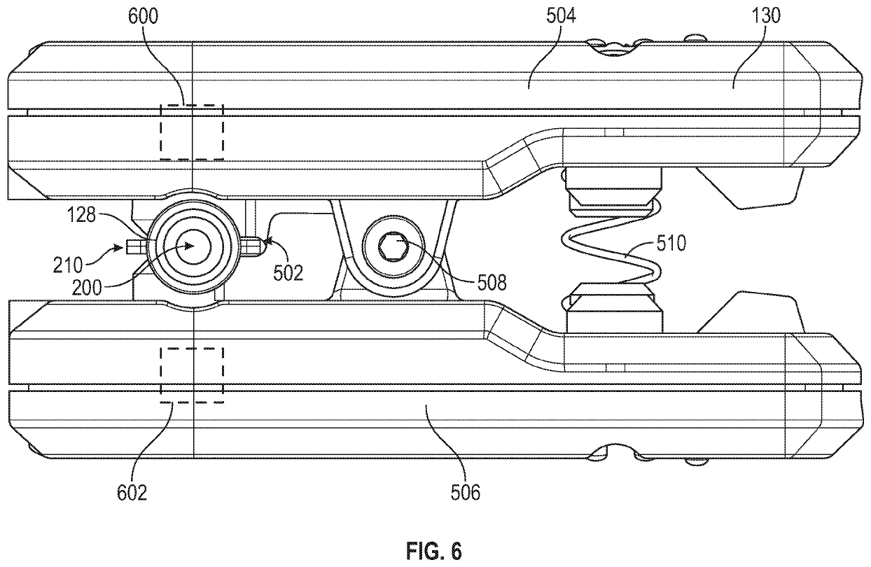

FIG. 4 illustrates a perspective view of another example of the cuvette 128, according to an embodiment of the present subject matter. As discussed herein, the cuvette 128 includes coupling features 210. In an example, the coupling features 210 include one or more wings 400. For instance, the one or more wings 400 may extend from the optical window 204. The wings 400 may facilitate alignment of the cuvette 128 (including the optical window 204) relative to the optical sensor 130 (shown in FIGS. 5 and 6).

FIG. 5 illustrates a side view of the cuvette 128 of FIG. 4 and the optical sensor 130, according to an embodiment of the present subject matter. As described herein, the blood filtration system 100 may include the optical sensor 130, and the optical sensor 130 may couple with the cuvette 128. For instance, the optical sensor 130 may include a cuvette socket 500 that receives the cuvette 128. In an example, the cuvette socket 500 includes coupling features 502 corresponding to the coupling features 210 of the cuvette 128. For example, the coupling features 502 of the optical sensor 130 may engage with the wings 400 to align the cuvette 128 relative to the optical sensor 130.

In another example, the optical sensor 130 includes a first portion 504 and a second portion 506. The cuvette socket 500 may be located between the first portion 504 and the second portion 506 of the optical sensor 130. The portions 504, 506 of the optical sensor 130 may engage with the cuvette 128 to retain the cuvette within the cuvette socket 500. For instance, a hinge 508 rotatably couples the first portion 504 with the second portion 506. The optical sensor 130 may include a biasing element 510, and the biasing element 510 may bias the first portion 504 toward the second portion 506, for instance to close the cuvette socket 500 around the cuvette 128. In another example, the cuvette socket 500 is located on a first side of the hinge, and the biasing element 510 is located on a second side of the hinge 508.

In an example, a coating may be applied to the cuvette 128, for example to enhance transmissive properties of light through the cuvette. For example, the coating may include (but is not limited to) a quarter wavelength coating coupled with the cuvette 128 to minimize reflectivity of the cuvette. Accordingly, the coating may facilitate transmission of light through the optical window 204 (e.g., reduce reflection of the light away from transmission through the optical window 204, or the like).

FIG. 6 illustrates another side view of the cuvette 128 of FIG. 4 and the optical sensor 130, according to an embodiment of the present subject matter. FIG. 6 shows the cuvette 128 received in the cuvette socket 500 of the optical sensor 130. As described herein, the first portion 604 of the optical sensor 130 is rotatably coupled with a second portion 506 of the optical sensor 130. The biasing element 510 may facilitate reception of the cuvette 128 by the cuvette socket 500 of the optical sensor 130 (also shown in FIG. 5). For example, the biasing element 510 facilitates engagement of the optical sensor 130 with the cuvette 128. For instance, the coupling features 210 of the cuvette 128 may engage with the coupling features 502 of the optical sensor (also shown in FIG. 5) to align the cuvette relative to the optical sensor 130.

In an example, optical sensor 130 includes one or more photoemitters 600 and one or more photoreceivers 602. The coupling features 210 of the cuvette 128 may cooperate with the coupling features 502 of the optical sensor 130 to align the optical window 204 (shown in FIG. 2) with the photoemitters 600 and the photoreceivers 602. Accordingly, the optical sensor 130 may transmit light with the photoemitters 600 through the optical window 204 (and the cuvette lumen 200 optionally containing fluid therein). The photoreceivers 602 may receive the light (or a portion of the light) from the optical window 204 generated by the photoreceivers 600. Thus, in an example, the photoemitters 602 may cooperate with the photoemitters 600 to measure one or more optical characteristics of the cuvette 128 (or fluid located therein).

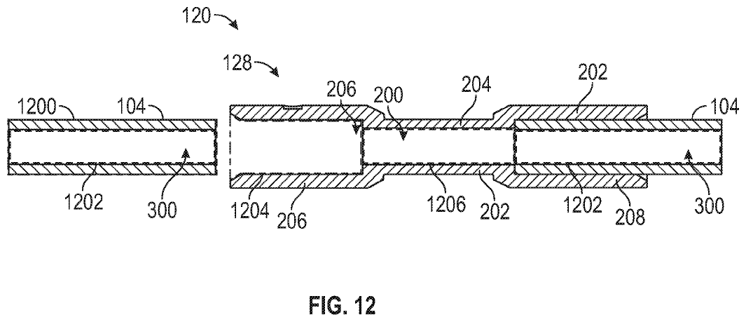

In another example, a profile (e.g., one or more of cross-section, shape, size, dimensions, contour, radius, perimeter, circumference, diameter, outline, boundary, configuration, pattern, arrangement, thickness, or the like) of the cuvette 128 (shown in FIG. 2) corresponds to a profile of one or more of the withdrawal line 104 or the infusion line 106. For example, a socket profile of the cuvette 128 may correspond to an outer diameter of the withdrawal line 104 (shown in FIG. 12), In another example, an inner diameter of one or more of the lines 104, 106 may correspond to an image distance (e.g., L in the BeerLambert law, the characteristic 302 shown in FIG. 3, or the like) of the system 100. Accordingly, the cuvette 128 may minimize disturbances in flow through the cuvette 128, such as by reducing one or more of eddies or shear forces in the flow of fluid through the cuvette 128 (and the blood circuit 120). For instance, clotting of blood in the cuvette 128 may be minimized because shear forces induced by the cuvette on the blood are reduced because of the correspondence in profiles between the cuvette 128 and one or more of the lines 104, 106. In yet another example, because the profile of the cuvette 128 corresponds with the profile of one or more of the withdrawal line 104 or the infusion line 106, the system 100 may correct for ambient light received by the photoreceiver, for instance because the cuvette 128 refrains from blocking ambient light (e.g., due to the profile correspondence with one or more of the lines 104, 106).

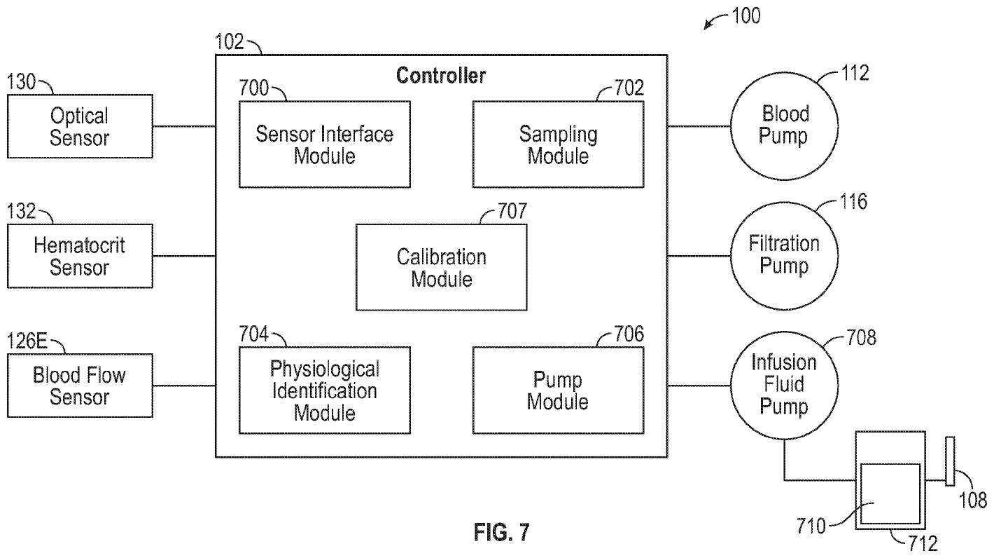

FIG. 7 illustrates a schematic view of another example of the blood filtration system 100, according to an embodiment of the present subject matter. As described herein, the blood filtration system includes the controller 102, the sensors 126, and one or more pumps, for instance the blood pump 112 and the filtration pump 116. In an example, the controller 102 includes one or modules, for instance a sensor interface module 700. The sensor interface module 700 may facilitate communication of the controller 102 with the sensors 126. In an example, the sensor interface module 700 receives one or more measurements from the sensors 126, such as receiving measured optical characteristics of the fluid in the blood circuit 120 from the optical sensor 130.

In another example, the controller 102 may include a sampling module 702. The sampling module 702 may record measurements received by the sensor interface module 700. For example, the sampling module 702 may record the optical characteristics measured by the optical sensor 130. For instance, the sampling module 702 may correlate measurements of the optical characteristics with respect to time. In one example, the sensor interface module 700 may receive optical characteristic measurements from the optical sensor 130, and sampling module 702 may record the optical characteristic measurements and associated timing for the optical characteristic measurements. In another example, the sampling module 702 may generate a data set including each optical characteristic measurement and a corresponding time when each optical measurement was taken.

Referring to FIG. 7, the controller 102 may include a physiological characteristic identification module 704 (“identification module 704”) that facilitates determining one or more physiological characteristics of a patient, such as plasma volume, hematocrit, red blood cell volume, or the like. The identification module 704 may determine one or more physiological characteristics based on measurements obtained by the blood filtration system 100, such as with the sensors 126. For instance, the identification module 704 may determine the plasma volume of the patient with the recorded optical characteristics. In an example, the identification module 704 may determine a concentration of an imaging substance in fluid within the blood circuit 120 based on optical characteristics of the fluid (e.g., optical characteristics measured with the optical sensor 130).

The identification module 704 may determine plasma volume of a patient based on the concentration of the imaging substance. For instance, the concentration of the imaging substance may correspond to the optical characteristics of the fluid. In an example, the concentration of the imaging substance is determined according to the Beer-Lambert law. For instance, the Beer-Lambert law may be expressed as:

I=I₀e^{-ϵCL}

where I is current provided by a photoreceiver (e.g., the photoreceiver 602, shown in FIG. 6). I₀ is current provided to a photoemitter (e.g., the photoemitter 600, shown in FIG. 6), c is a molar absorption coefficient of a substance (e.g., one or more plasma constituents, ICG, or the like). In an example, L is the distance between the photoemitter 600 and the photoreceiver 602. In another example, L corresponds to the first characteristic 302 of the cuvette 128. In yet another example, C is the concentration of the substance in the fluid.

In still yet another example, the sensor interface module 700 may provide a specified current to the photoemitter 600 (shown in FIG. 6), and the photoemitter 600 may generate light at a specified wavelength and a specified intensity when the photoemitter 600 receives the specified current. The photoemitter 600 may transmit light to the photoreceiver 602, For instance, the photoemitter 600 may transmit light through the optical window 204 of the cuvette 128, The photoreceiver 600 may receive the light transmitted through the optical window 204 by the photoemitter 600. For instance, the light transmitted by the photoemitter 600 may be shifted (e.g., attenuated, changed, altered, or the like) as it is transmitted through the optical window 204. Accordingly, the photoreceiver 600 may receive light at one or more of a shifted wavelength and a shifted intensity. The shifted wavelength may be different than the specified wavelength. The shifted intensity may be different than the specified intensity. Thus, the photoreceiver 602 may provide an output current (e.g., I, or the like) different than the specified current (e.g., I₀, or the like) provided to the photoemitter 600.

As described herein, the Beer-Lambert law includes the variable c, corresponding to a molar absorption coefficient of a substance, for example an imaging substance (e.g., ICG, or the like) in fluid within the cuvette 128 (shown in FIG. 2). For example, indocyanine green has a molar absorption coefficient of 0.2 cm⁻¹/μM. Accordingly, the blood filtration system 100 may determine the concentration of the imaging substance in fluid within the blood circuit 120 according to the Beer-Lambert law, for instance because the concentration may be determined using known (or measured) variables (e.g., one or more of molar absorption coefficient of a substance, the first characteristic 302 of the optical window 204, and the current provided by the photoreceiver 602). Thus, the blood filtration system 100 provides enhanced accuracy (or precision) of imaging substance concentration determinations in comparison to a healthcare provider manually drawing blood samples from a patient because the optical properties of the blood may be measured within the blood circuit 120, and at a rate significantly greater than what may be obtained by manually drawing blood samples and determining the concentration of imaging substance within the blood samples withdrawn from vasculature of a patient (e.g., optical properties of a blood sample contained in a vial).

The physiological characteristic identification module 704 may determine a plasma volume of a patient, for instance based on the determined concentration of an imaging substance in fluid within the blood circuit 120. For example, plasma volume of a patient may be expressed as:

$$Plasma Volume = \frac{Specified Quantily of Infused Imaging Substance}{Concentration of Imaging Substance}$$

In an example, a specified quantity of imaging substance may be infused into the blood circuit 120 (e.g., using the infusion fluid pump 708, or the like). In another example, the specified quantity of the imaging substance may be infused into vasculature of a patient. The imaging substance mixes with blood in the blood circuit 120 (and vasculature), and is cleared with one or more physiological processes (e.g., removal of ICG by the liver, or the like). In this example, because the specified quantity of imaging substance is infused into vasculature space of a patient (and mixed with the blood therein), the optical characteristics of the blood vary in correspondence with the concentration of the imaging substance in the vascular space. The blood filtration system 100 measures the optical characteristics of the blood (containing imaging substance therein), such as with the optical sensor 130. The identification module 704 may determine the concentration of the imaging substance in the blood. The identification module 704 may determine the plasma volume based on the specified quantity of imaging substance infused into the patient and the determined concentration of the imaging substance. Accordingly, the blood filtration system 100 may determine the plasma volume of a patient while a healthcare provider refrains from drawing blood samples from the patient. Thus, the blood filtration system 100 enhances accuracy (or precision) of plasma volume determinations, for instance because optical characteristics are measured multiple times a second (in contrast to a healthcare provider manually collecting a blood sample over the course of, for example, 15 seconds).

In another example, the identification module 704 may determine the plasma volume based on changes in hematocrit of the patient. For instance, the system 100 may determine the plasma volume at a first time (e.g., the beginning of therapy, or the like). The system 100 may monitor changes in the hematocrit value of a patient, and accordingly monitor changes in plasma volume. For example, because the system 100 may determine the red blood cell volume of a patient, and the red blood cell volume may remain constant (or nearly constant) during therapy, the changes in hematocrit may be used to determine changes in plasma volume, for instance while refraining from injecting more than one infusion volume of the imaging substance. Accordingly, the system 100 may determine the plasma volume of a patient, for example by monitoring changes in the hematocrit value of the patient.

Referring to FIG. 7, the blood filtration system includes one or more pumps, for instance an infusion fluid pump 708. In an example, the controller 102 includes a pump module 706, and the pump module 706 modulates the one or more pumps of the system 100. For instance, the pump module 706 may modulate a speed, flow rate, output, or the like of the one or more pumps, for instance to pump fluid within the system 100. In another example, the infusion fluid pump 708 may facilitate infusion of a fluid, for example an imaging substance 710 into the blood circuit 120. In an example, the imaging substance 710 includes one or more of indocyanine green, methylene blue, a radioactive tracer, or the like. For instance, the imaging substance 710 may include indocyanine green bound to albumin. The imaging substance 710 may be contained within an infusion container 712 (e.g., vessel, bag, or the like). The pump module 706 may modulate the infusion fluid pump 708, for example to adjust the speed of the infusion pump 708. In an example, the pump module 706 modulates the infusion pump 708 to infuse a specified quantity of the imaging substance 710. In an example, the infusion fluid pump 708 pumps the imaging substance 710 from container 712 and into one or more of the access ports 122 (shown in FIG. 1), accordingly, the infusion fluid pump 708 facilitates infusion of the imaging substance 710 into the blood circuit 120. In another example, the infusion container 712 may be in communication with the catheter 108. The infusion pump 708 may pump the imaging substance 710 from the container 712 and into the catheter 108. Accordingly, the blood filtration system 100 may facilitate infusion of a specified quantity of the imaging substance 710 into the blood circuit 120. Thus, the blood filtration system 100 may facilitate infusion of a specified quantity of the imaging substance 710 into vasculature of a patient.

In an example, the pump module 706 may adjust the speed of the filtration pump 116 in correspondence with the infusion pump 708. For instance, the pump module 706 may stop the filtration pump 116 during operation of the infusion fluid pump 708. In another example, the pump module may stop the filtration pump 116 for a specified duration (e.g., 3 minutes, 10 minutes, 15 minutes, or the like) after modulating the infusion fluid pump 708 to infuse the specified volume (or infused volume) of imaging substance 710 into the blood circuit 120. Accordingly, the blood filtration system may minimize removal of the imaging substance from the fluid in the blood circuit 120 during measurement of optical properties of the fluid in the blood circuit 120. Further, stopping the filtration pump 116 for the specified duration after infusion of the imaging substance 710 may enhance accuracy (or precision) of plasma volume determinations, for instance by stopping removal of plasma constituents while the system determines the plasma volume of the patient. Thus, changes in plasma volume may be minimized while the system 100 determines the plasma volume of the patient (e.g., using the identification module 704, or the like). Accordingly, the system 100 may refrain from removing one or more plasma constituents with the filter 110 while determining the plasma volume of the patient, for instance to enhance the accuracy of the plasma volume determination.

The blood filtration system 100 may monitor a total latency duration between infusion of the imaging substance (e.g., with the pump module 706 modulating the infusion fluid pump 708) and detection of the imaging substance 710 by the optical sensor 130. For instance, the system 100 monitors the total latency duration to compensate for the total latency duration in plasma volume determinations. In an example, the infusion fluid pump 708 infuses a specified quantity (e.g., 5 milliliters, 7.4 milliliters, 20 milliliters, or the like) into vasculature of the patient at a specified time value, such as an initial infusion time T₀. For instance, the initial infusion time T₀ may correspond to when the pump module 706 modulates the infusion fluid pump 708 and accordingly infuse the specified about of the imaging substance 710. The specified quantity of the imaging substance 710 may mix with the intravascular space of the patient (and the fluid, such as blood, contained therein). As the imaging substance 710 mixes with fluid in the intravascular space, the concentration of the imaging substance correspondingly increases. The fluid (including the imaging substance 710) may flow into the blood circuit 120, and the optical sensor 130 may measure the optical characteristics of the fluid. As discussed herein, the system 100 may determine the concentration of the imaging substance 710 in the fluid within the blood circuit 120. The total latency duration may correspond to a time differential between infusion of the imaging substance 710 (e.g., T₀, or the like) and when the concentration of the imaging substance 710 exceeds a specified concentration threshold (e.g., 0.05 percent, 4 percent, or the like). Accordingly, the identification module 704 may compare the concentration of the imaging substance 710 to the specified concentration threshold. The sampling module 702 may record the total latency duration, for example the difference in time between infusion of the imaging substance 710 and the concentration of the imaging substance 710 exceeding the specified concentration threshold. In an example, the system 100 compensates for circulation of the imaging substance 710 prior to mixing with the fluid in the intravascular space of the patient (and the blood circuit 120), For instance, the catheter 108 may be located in vasculature of a patient, and the imaging substance may be infused into the vasculature of the patient. The imaging substance may flow from an infusion lumen of the catheter to a withdrawal lumen of the catheter without fully mixing with the intravascular spaced (e.g., by flowing to the heart and then flowing to the withdrawal lumen, or the like). In another example, the sampling module 702 refrains from recording optical characteristics of the fluid in the blood circuit 120. For instance, the sampling module 702 may refrain from recording optical characteristics for a specified duration (e.g., 5 seconds, 12 seconds, 90 seconds, or the like) after infusion of the imaging substance. Accordingly, the system 100 may compensate for recirculation of the imaging substance from the infusion lumen to the withdrawal lumen prior to mixing with the intravascular space of a patient. In another example, the system 100 may include a valve, and the controller 102 may modulate the valve to inhibit flow in the withdrawal lumen for a specified duration after infusion of the imaging substance, for instance to minimize recirculation into the withdrawal lumen.

In another example, the system 100 (e.g., the controller 102 including the identification module 704, or the like) may monitor the total latency duration (e.g., the latency duration 804, or the like) to compensate for the amount of time required for the infused imaging substance to circulate through the blood circuit 120 (in contrast to the circulating through the intravascular space of the patient and the blood circuit 120). For instance, the blood filtration system may include a plurality of optical sensors, and the plurality of optical sensors may be coupled to the blood circuit at a plurality of respective locations In one example, a first optical sensor may be coupled with the blood circuit 120 before the filter 110 (e.g., by coupling with a cuvette included in the withdrawal line 104, or the like). In yet another example, a second optical sensor may be couple with the blood circuit after the filter 110 (e.g., by coupling with a cuvette included in the infusion line 106, or the like).

In an example, the controller 102 is in communication with the optical sensors, and a calibration module 707 of the controller 102 may monitor a circuit latency duration. For example, the circuit latency duration may include a time differential between the first optical sensor detecting the imaging substance and the second optical sensor detecting the imaging substance. Accordingly, the calibration module 707 may determine the time for the infusion substance to circulate through the blood circuit 120 (or a portion of the blood circuit 120). Thus, the controller 102 may monitor the circuit latency duration, for instance to determine an intravascular latency duration. In an example, the intravascular latency duration may include a time differential for the imaging substance to circulate the intravascular space. The controller (e.g., calibration module 707, or the like) may determine the intravascular latency duration by determining the difference between the total latency duration and the circuit latency duration. Accordingly, the system 100 may compensate for latency durations while determining physiological characteristics of a patient, such as plasma volume or red blood cell volume.

In another example, when the filtration pump 116 is operated (e.g., when the extraction rate is greater than zero, or the like), the blood in the circuit 120 after the filter 110 (shown in FIG. 1) may become hemo-concentrated (which may raise the hematocrit within the filter 110). The magnitude of this rise may be determined from the blood flow rate and extraction rate of the system 100 in conjunction with various physiological characteristics e.g., a determined hematocrit value at the beginning of therapy).

In another example, the system 100 may facilitate clearance of the imaging substance, for example by infusing a fluid, such as saline (that does not contain the imaging substance). For instance, the infusion fluid pump 708 may infuse saline into the patient while the filter 110 remove plasma constituents (including water) from blood in the filter 110. The plasma constituents may include the imaging substance. Accordingly, the system 100 may remove the imaging substance with the filter. Optionally, the system may remove the imaging substance while maintaining the plasma volume of the patient (e.g., by infusing saline in correspondence with removal of the plasma constituents with the filter 110). In another example, the system may determine the plasma volume of the patient based on the extraction rate after a specified time and a ratio of the concentrations of the imaging substance after the specified time period.

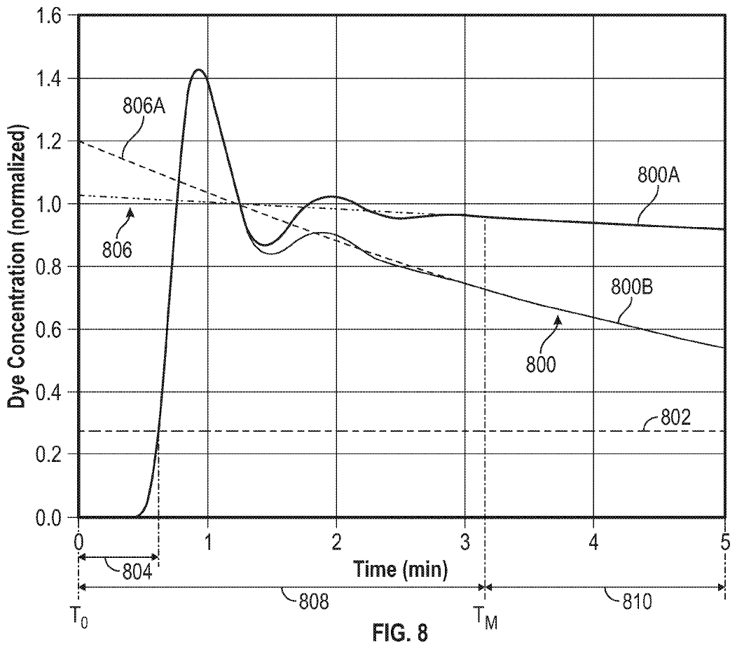

Referring to FIG. 7, and as described herein, the controller 102 may include the calibration module 707. The calibration module may compare measured optical characteristics to an optical characteristic threshold. In another example, the calibration module 707 may measure a latency duration, such as a total latency duration, between when the pump module modulates the infusion pump and the when the measured one or more optical characteristics exceed the optical characteristic threshold. In yet another example, the calibration module 707 may measure a latency duration between when the pump module modulates the infusion pump and when a determined concentration of an imaging substance exceeds a concentration threshold (e.g., the concentration threshold 802 shown in FIG. 8, or the like). In still yet another example, the calibration module 707 may compare the latency duration to a latency threshold. For instance, the sampling module may retrain from recording optical characteristics for a specified time period when the latency duration exceeds the latency threshold.

Referring to FIG. 7, the controller 102 may determine a filtration fraction of the system 100. For instance, the filtration fraction can include a ratio of a filtration rate (e.g., a rate that one or more plasma constituents is extracted from the filter) to a blood flow rate through the filter. The pump module 706 may control a speed of one or more pumps to adjust the filtration fraction of the system 100. The controller can vary the speed of the one or more pumps (e.g., the filtration pump, the blood pump, or the like) to adjust the filtration fraction. Additionally, the controller can be configured to determine the hematocrit value, and can compare the hematocrit value to a hematocrit threshold. The controller can be configured to maintain the filtration fraction, for instance when the hematocrit value equals the hematocrit threshold. Further, the controller can be configured to adjust the filtration fraction, for instance if the hematocrit value exceeds, or declines below, the hematocrit threshold. In some examples, the hematocrit threshold is a range of hematocrit values (e.g., 45 percent to 55 percent). Controlling the filtration fraction can be used to control the rate of removing plasma constituents while maintaining preferred patient diagnostic parameters. Additionally, controlling the filtration fraction can be used to reduce clogging of the extracorporeal components of a blood filtration system. For instance, when the hematocrit value is high (e.g., greater than 50 percent) the filter can have an increased probability of clogging). In some examples, the filtration fraction can be reduced (e.g., filtration rate reduced, blood flow rate increased, or a combination thereof) to reduce the probability of clogging in the filter.

As described herein, the controller 102 may determine a filtration fraction of the system 100. The filtration fraction may be expressed as:

$$Filtration Fraction = \frac{Filtration Fluid Extraction Rate}{( 1 - HCT Value ) ( Blood Flow Rate )}$$

In an example, the hematocrit (“HCT”) value is compared to a hematocrit threshold (e.g., 0.50) and if the hematocrit value is greater than the hematocrit threshold, or if the filtration fraction is greater than a filtration fraction threshold (e.g., 0.20), the filtration fraction can be reduced at operation 360. Reducing the filtration fraction can be reduced, for instance, by the controller 102. The controller can vary the speed, flow rate, output, or the like of the one or more pumps (e.g., the filtration pump 116, the blood pump 112, or the like) to adjust the filtration fraction. Controlling the filtration fraction can be used to control the rate of removing plasma constituents while maintaining preferred patient diagnostic parameters. Additionally, controlling the filtration fraction can be used to reduce clogging of the extracorporeal components of a blood filtration system (e.g., the blood filtration system 100, or the like), In this example, if the hematocrit value is less than the hematocrit threshold, or if the filtration fraction is less than a filtration fraction threshold (e.g., 0.20), the blood flow rate of the system can be set to a user set blood flow rate (e.g., operating the blood pump 112 at the first blood flow rate).

FIG. 8 illustrates a graphical representation of a plot including a concentration (e.g., along a y-axis of the plot, or the like) of an imaging substance with respect to time (e.g., along an x-axis of the plot, or the like), according to an embodiment of the present subject matter. As described herein, the controller 102 may determine the concentration of an imaging substance based on measured optical characteristics of fluid in the blood circuit. In an example, the physiological characteristic identification module 704 may repeatedly determine the concentration of an imaging substance (e.g., ICG, or the like). The sampling module 702 may record the concentration of the imaging substance, for example to provide a concentration data set 800 including values corresponding to the determined concentration of imaging substance recorded with respect to time. In one example, the sampling module 702 may associate a value corresponding to each imaging substance concentration determination with a respective time for when the specific imaging substance concentration are determined (or when optical characteristics corresponding to the concentration of the imaging substance are measured).

In an example, FIG. 8 shows a first concentration data set 800A and a second concentration data set 800B. The first concentration set 800A may correspond to determined concentrations of an imaging substance where the imaging substance is cleared by physiological processes at a first clearance rate (e.g., clearance of 5 percent of the imaging substance per minute, or the like). The second concentration set 800B may correspond to determined concentrations of an imaging substance where the imaging substance is cleared by physiological processes at a second clearance rate (e.g., clearance of 10 percent of the imaging substance per minute, or the like).

As described herein, the physiological characteristic identification module 704 (shown in FIG. 7) may compare the concentration of the imaging substance 710 (e.g., shown in FIG. 8 as 800A or 800B) to a concentration threshold 802. In an example, the identification module 704 comparing of the concentration of the imaging substance 710 to the concentration threshold 802 facilitates determining the concentration of the imaging substance 710 in the intravascular space of the patient after infusion of the imaging substance 710. For example, the imaging substance 710 may mix with the intravascular space when a portion of the imaging substance infused into the patient flows through a heart of a patient, flows back to the blood circuit 120 and is detected by the optical sensor 130 (e.g., by measuring optical characteristics of the fluid in the blood circuit 120). Accordingly, the blood filtration system 100 may enhance accuracy of plasma volume determinations by refraining from recording concentrations that refrain from exceeding the concentration threshold 802. Thus, the blood filtration system may compensate for recirculation between the infusion lumen and the withdrawal lumen (e.g., without mixing in the intravascular space) by refraining from recording concentrations (or optical characteristics) that exceed the concentration threshold 802 (or an optical characteristic threshold, such as a minimum intensity level, or the like).

In another example, the identification module 704 may determine a latency duration 804 between infusion of the imaging substance 710 and detection of the imaging substance 710 at a concentration above the concentration threshold 802. For instance, the identification module 704 may determine the latency duration 804 by establishing a time differential between infusion of the imaging substance (e.g., T₀, or the like) and detection of the imaging substance after mixing with the intravascular space (e.g., when the concentration of the imaging substance 710 exceeds the concentration threshold 802). Accordingly, the blood filtration system may determine a base time for the imaging substance 710 to mix with the patient fluid in the intravascular space. Thus, the accuracy (or precision) of plasma volume determinations by the system is enhanced because the system 100 may compensate for the latency duration 804 (and associated mixing of the imaging substance 710) in contrast to potential distortions in plasma volume determinations had the latency is not counted. For instance, the determining the latency duration may facilitate extrapolation along the decay function to determine the extrapolated concentration. In one example, the determining the latency duration facilitates extrapolating along the decay function within the mixing period where the imaging substance is mixing with intravascular space of a patient. Further, the identification module 704 may determine cardiac output of a patient, for instance by integrating (e.g., with a mathematical operation, or the like) the concentration data set with respect to time to determine a first cardiac output value, and dividing the specified quantity of imaging substance 710 (infused into a patient) by the first cardiac output value. In yet another example, the identification module may determine total blood volume of a patient by multiplying (e.g., with a mathematical operation, or the like) the cardiac output by the latency duration 804.

In an example, the identification module 704 fits the concentration data set to a curve, for instance to extrapolate along the curve and determine an extrapolated concentration 806 of the imaging substance 710 in the blood circuit 120 (and the intravascular space in communication with the blood circuit 120). The extrapolated concentration 806 may enhance the accuracy of plasma volume determinations, for instance by correcting for physiological processes, such as clearance, that affect the measured optical characteristics (and corresponding concentration of the imaging substance 710) of fluid in the blood circuit 120 (and intravascular space). For example, the concentration of the imaging substance 710 in fluid within the intravascular space may vary during a mixing period 808 as the imaging substance 710 mixes with fluid in the intravascular space. The extrapolated concentration corrects for physiological processes by extrapolating along the curve to determine a concentration of the imaging substance 710 within the mixing period 808, In an example, the extrapolated concentration provides an estimate (due to extrapolation) of the concentration of the imaging substance 710 as if it instantly mixed with the fluid in the intravascular space. In this example, because mixing of the imaging substance 710 with fluid in the intravascular space is not instantaneous, extrapolating along the curve to determine the extrapolated concentration may enhance accuracy (or precision) of concentration determinations because the extrapolated concentration provides an estimate of the concentration of imaging fluid as if instantly mixed. Accordingly, the extrapolated concentration 806 may enhance accuracy of the plasma volume determination because the extrapolated concentration 806 may correct for physiological and mechanical processes (e.g., fluid mechanic mixing of the imaging substance with fluid in the intravascular space) and effects of the system 100 (e.g., the latency duration 804, or the like) that may distort the measured concentration of the imaging substance in the fluid determined by the system 100. For instance, the estimation provided by the extrapolated concentration 806 enhances accuracy of the plasma volume determination because the concentration of imaging substance 710 in the fluid starts diminishing, for example due to clearance of the imaging substance 710 by the liver of the patient. Accordingly, the extrapolated concentration 806 enhances accuracy of plasma volume determinations by enhancing the accuracy of determining the concentration of the imaging substance in the intravascular space (and the fluid therein). Thus, in an example the extrapolated concentration 806 facilitates determination of the concentration of the imaging substance in intravascular space based on one or more of the time of infusion of the imaging substance and the time the measurement is desired or requested.

In an example, polynomial (e.g., including one or more inflection points) portions of the curve (fit to the determined concentration of imaging substance) shown in FIG. 8 are within the mixing period 808. The mixing period 808 may be located between T₀ and T_{M} (shown in FIG. 8). For instance, T_{M} may correspond to a point where portions of the curve (or function) become linear. In another example, linear portions of the curve are within a decay period 810. The decay period may be located beyond T_{M} (shown in FIG. 8 as extending toward the five minute edge of the plot). Extrapolating along the curve facilitates determining the plasma volume within the mixing period 808. Determining the plasma volume within the mixing period 808 increases the accuracy (or precision) of plasma volume determinations with the system 100, for instance because extrapolating along the curve corrects for physiological (and system) effects that may obscure the concentration.

In another example, the identification module 704 may determine the extrapolated concentration 806 (e.g., the concentration 806A, or the like) based on one or more of extrapolation, regression analysis, interpolation, or the like. For instance, the concentration of the imaging substance 710 (or measured optical characteristics of fluid in the blood circuit) may be fit to a function (e.g., a curve, line or the like), for instance using a least means squares regression fit. In an example, the determined concentration of the imaging substance 710 may be fit to a mono-exponential decay curve, for instance with the identification module 704 using a least mean squares regression fit on the concentration data set. The identification module 704 may extrapolate along the mono-exponential decay curve to determine the concentration (e.g., extrapolated concentration 806A, or the like) of the imaging substance 710 proximate to the time of injection (e.g., a time within the mixing period 808, or the like). For instance, the concentration is determined by extrapolating along the mono-exponential decay curve to a time within the mixing period 808, the determined concentration of the imaging substance 710 may be fit to a bi-exponential decay curve. FIG. 8 shows the first extrapolated concentration 806A extrapolated from the second concentration data set 800B.

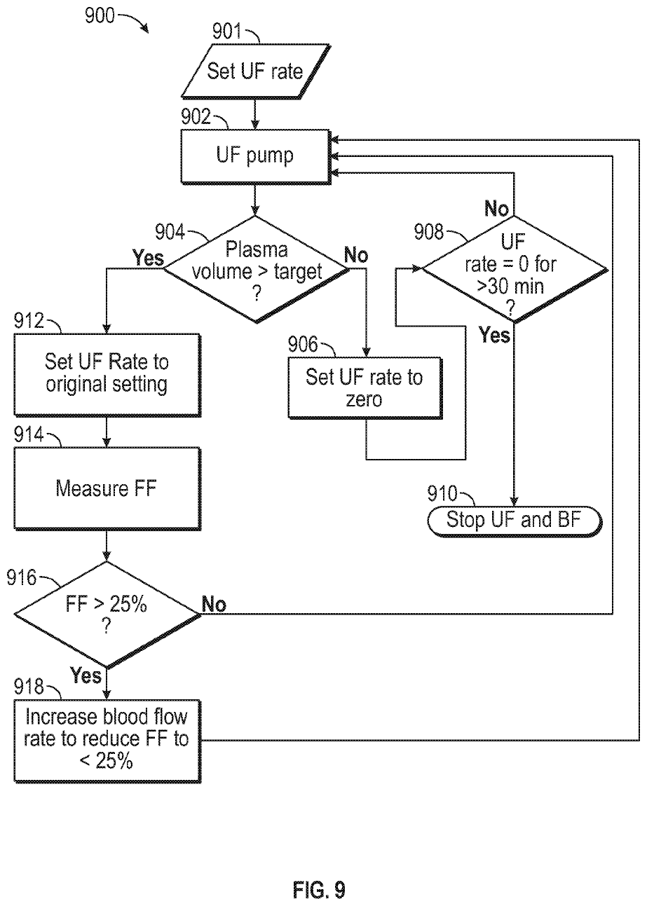

FIG. 9 illustrates a diagram of an example of a method 900 for removing one or more plasma constituents from fluid in vasculature of a patient, according to an embodiment of the present subject matter. In describing the method 900, reference is made to one or more components, features, functions, operations, apparatuses, and systems previously described herein. Where convenient, reference is made to the components, features, operations and the like with reference numerals. The reference numerals provided are exemplary and are not exclusive. For instance, components, features, functions, operations, apparatus, systems, and the like described in the method 900 include, but are not limited to, the corresponding numbered elements provided herein and other corresponding elements described herein (both numbered and unnumbered) as well as their equivalents.

In an example, the method 900 may include at 901 an extraction rate may be set to a specified extraction rate, for instance by a healthcare provider or the controller 102 (e.g., with the pump module 706 adjusting a flow rate of the filtration pump 116). Accordingly, at 902 the filtration pump 116 may operate at the set extraction rate. The method 900 may include at 904 comparing the plasma volume of a patient to a target plasma volume. For instance, the physiological characteristic identification module 704 may compare the determined plasma volume of the patient to the target plasma volume. At 906, the extraction rate (e.g., rate of removing one or more plasma constituents with the filter 110) may be set to zero. For instance, the flow rate of the filtration pump 116 may be set to zero, or no flow. In an example, the extraction rate may be set to zero when the plasma volume (e.g., as determined by the identification module 704) is less than (or equal to) the target plasma volume.

At 908, the controller 102 (such as pump module 706) may monitor the plasma volume of a patient. In another example, at 910 the method 900 may include setting the flow rate through the blood circuit 120 to zero. For instance, the pump module 706 may stop the blood pump 112 and the infusion pump when the determined plasma volume of the patient is less than (or equal to) the target plasma rate for a specified duration of time, such as 30 minutes or the like. Accordingly, the blood filtration system 100 may determine when to end therapy of the patient, for instance when the plasma volume of the patient is at (or below) the target plasma volume.

Referring to FIG. 9, the method 900 may include at 912 modulating the filtration pump 116 to operate the pump 116 at the specified extraction rate. For instance, the pump module 706 may operate the pump 116 at the specified extraction rate when the determined plasma volume of the patient is greater than the target plasma volume of the patient. For instance, the controller 102 may monitor the plasma volume of the patient once the plasma volume meets the target plasma volume. The controller 102 may continue monitoring the plasma volume of the patient (e.g., for 10 minutes, 30 minutes, an hour, or the like) after meeting the target plasma rate (with the extraction rate set to zero), for instance to determine if additional fluid is recruited from tissue into the intravascular space. In an example, the pump module 706 may increase the extraction rate above zero, for instance when the plasma volume of the patient exceeds the plasma target. Accordingly, the system 100 may guide blood filtration therapy with the controller 102, for instance to maintain the plasma volume of the patient at the target plasma rate.

In an example, the system 100 modulates the one or more pumps in proportion to the difference between the determined plasma volume of a patient and the target plasma volume for the patient. In another example, the system 100 may control the one or more pumps according to one or more of a proportional control scheme, a proportional-integral control scheme, or a proportional-integral-derivative control scheme—based on the difference between the determined plasma volume of the patient and the target plasma volume of the patient. For instance, the pump module 706 may operate the filtration pump 116 at a first flow rate to extract one or more plasma constituents from fluid in the filter 110 at a first extraction rate. As the difference between determined plasma volume and the target plasma volume decreases, the pump module 706 may correspondingly decrease the extraction rate of the system 100, for example to a second flow rate (less than the first flow rate). Accordingly, as the determined plasma volume of the patient approaches the target plasma volume, the system 100 may decrease the rate that plasma constituents are removed (and correspondingly the rate that plasma volume is decreasing). Thus, performance of the blood filtration system 100 is enhanced, for example because the system 100 may refrain from removal of too many plasma constituents (e.g., water, or the like) from the patient.

At 914, the method 900 may include determining a filtration fraction (“FF”) of the system 100. For instance, the controller 102 (such as the pump module 706) may determine the filtration fraction of the system. In an example, the filtration fraction can include a ratio of the extraction rate (e.g., a rate that one or more plasma constituents is extracted from the filter 110, or the like) to a blood flow rate through the filter 110 (e.g., the rate of pump 112, or the like). The controller 102 may monitor the filtration fraction, for instance to decrease clotting in the blood circuit 120 (including the filter 110). For instance, at 918, the controller 102 may increase the blood flow rate when the filtration fraction is greater than 0.25. In another example, the controller 102 (such as the pump module 706) may decrease the extraction rate to reduce the filtration fraction when the filtration fraction is greater than 0.25.

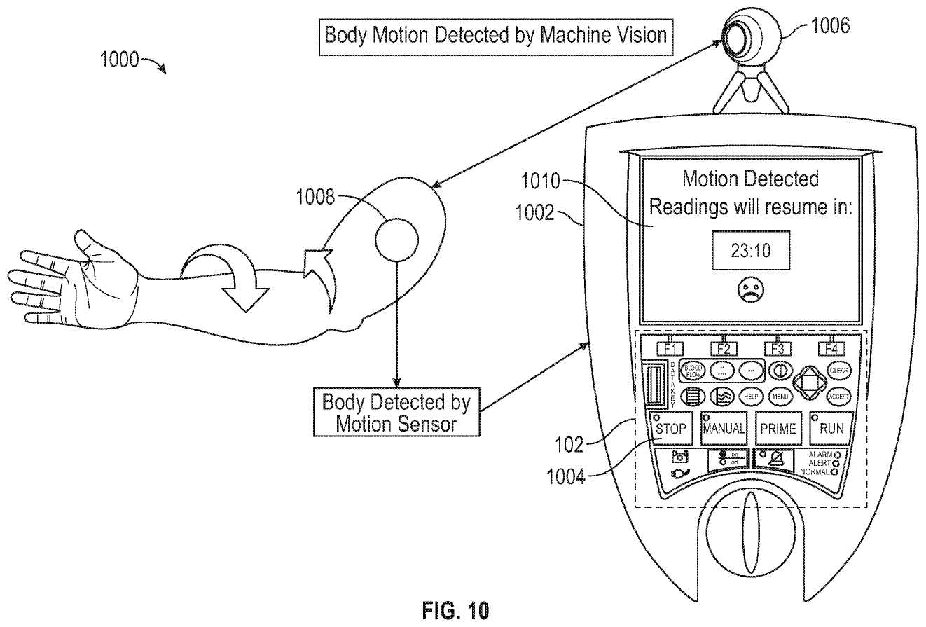

FIG. 10 illustrates a schematic view of another example of the filtration system 100, according to an embodiment of the present subject matter. The blood filtration system 100 may include a system housing 1002 that includes controls 1004 (e.g., buttons) that may change various operating parameters of the system 100, for instance a button that operates the blood pump 112 (shown in FIG. 1). The system housing 1002 may include the controller 102, and the controls 1004 may communicate with the controller 102 to operate one or more components of the blood filtration system (e.g., the filtrate pump 116 shown in FIG. 2).

The blood filtration system 100 may include an optical sensor 1006 (e.g., a camera, an infrared camera, or the like) that may monitor movement of the patient, for instance my observing a patient reference point 1008 (e.g., a head of the patient, a mark included on a surface of an object secured to the patient, for instance a blood pressure cuff, or the like). The optical sensor 1006 may monitor the position of the patient reference point and the controller 102 may be configured to determine if movement of the patient from an initial position affects the determined hematocrit value of the patient. The hematocrit value determination (or other physiological characteristics, such as plasma volume or the like) may be affected by movement of a patient, for example when the patient transitions from a supine position (e.g., laying down) to a standing position. Thus, as the system 100 removes plasma constituents (e.g., water, or the like) from a patient and accordingly reduces a plasma volume of the patient, it may be difficult to differentiate between a reduction in the plasma volume due to purposeful therapy with the system 100 and volume reduction due to patient postural changes. Accordingly, the system 100 may monitor postural changes of a patient, for instance to determine if the postural changes affect one or more physiological characteristic determinations by the identification module 702 (shown in FIG. 7), such as a plasma volume determination or a hematocrit value determination.

In an example, the controller 102 may provide a notification if the hematocrit (or other physiological characteristic) determination is affected by the movement of the patient. For instance, the controller 102 may provide a notification on a display 1010 with a timer indicating the time since the patient moved in a way that affected the hematocrit value determination. In an example, the controller 102 may compare the change in position of the portion of the body of the patient to a positional threshold and provide a notification when the change in position exceeds the positional threshold. For instance, the controller 102 may determine the position of the patient reference point 1008 relative to the optical sensor 1006.

In yet another example, the controller 102 may operate a speaker and change a tone according to the patient movement (e.g., initiate a beep if the patient moves). Additionally, the controller 102 may determine a movement value for instance a difference between the position of the patient reference point to the initial position. Further, the controller 102, such as the physiological characteristic identification module 704, may compare the movement value to a movement threshold and may provide a notification if the movement value exceeds the movement threshold.

In another example, the controller 102 may refrain from providing a notification of the hematocrit value (or other physiological characteristic) when the hematocrit value (or other physiological characteristic) is affected by movement of the patient (e.g., by refraining from displaying the movement-affected hematocrit value on the display 1010). Accordingly, the blood filtration system 100 may provide additional information (e.g., to a healthcare provider) that the hematocrit value of the patient has been affected by movement of the patient.

In yet another example, the patient reference point 1008 may include an accelerometer, and the accelerometer may be in communication with the controller 102 (e.g., with a wired or wireless communication pathway). The accelerometer may monitor the movement of the patient by determining acceleration of a portion of a body of the patient (e.g., an arm). The controller 102 may determine if the movement of the patient from the initial position affects the determined hematocrit value of the patient, for instance by comparing the acceleration of the portion of the body of the patient to an acceleration threshold.

In still yet another example, a pressure sensor in communication with a blood stream of the patient (e.g., the first sensor 124A or the second sensor 124B shown in FIG. 1) may monitor a change in pressure within the blood stream of the patient. The controller 102 may determine if the movement of the patient from the initial position affects the determined hematocrit value of the patient, for instance by comparing the change in pressure (e.g., because the patient transitioned from laying down to standing) within the blood stream to a pressure threshold. The controller 102 may provide a notification if the change in pressure exceeds the pressure threshold.

In some examples, the controller 102 may apply a correction value to the determined hematocrit value of the patient according to the movement of the patient. For instance, the correction value may be determined by evaluating a change in the hematocrit value according to the motion of the patient. The controller 102 may log data associated with changes in patient position and corresponding changes in hematocrit values to determine the correction value. For instance, the sampling module 702 may record data associated with changes in patient position and corresponding changes in physiological characteristics, including (but not limited to) plasma volume, hematocrit, cardiac output, or the like.

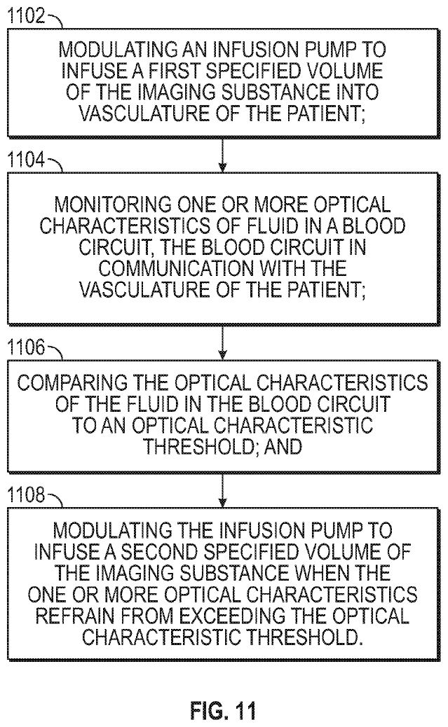

FIG. 11 illustrates one example of a method 1100 for administering a patient-specific imaging substance dosage, according to an embodiment of the present subject matter. In describing the method 1100, reference is made to one or more components, features, functions, operations, apparatuses, and systems previously described herein. Where convenient, reference is made to the components, features, operations and the like with reference numerals. The reference numerals provided are exemplary and are not exclusive. For instance, components, features, functions, operations, apparatus, systems, and the like described in the method 1100 include, but are not limited to, the corresponding numbered elements provided herein and other corresponding elements described herein (both numbered and unnumbered) as well as their equivalents.

In an example, at 1102, the method 1100 includes infusing a first specified volume of an imaging substance 710 into a blood circuit 120, for example with the infusion pump 708. For instance, the blood circuit 120 may include a catheter 108, and the catheter 108 may facilitate infusion of the imaging substance into vasculature of a patient. In an example, the pump module 706 may modulate the infusion pump 708 to pump one or more of the first specified volume or the second specified volume.

At 1104, the method 1100 includes monitoring one or more optical characteristics of fluid in the blood circuit 120. For example, the optical characteristics may be monitored to determine a concentration of the imaging substance within the fluid. The method 1100 may include at 1106 comparing the optical characteristics of the fluid in the blood circuit to an optical characteristic threshold, instance, the calibration module 707 may compare the measured optical characteristics to the optical characteristic threshold. The calibration module 707 may determine if the optical characteristics meet the optical characteristic threshold, for instance meeting the optical characteristic threshold within a first latency duration.

At 1108, the method 1100 includes modulating the infusion pump to infuse a second specified volume of the imaging substance when the one or more optical characteristics refrain from exceeding the optical characteristic threshold. For instance, the second specified volume may be infused when the one or more optical characteristics refrain from exceeding the optical characteristic threshold within a latency duration, such as the first latency duration. In another example, the calibration module 707 may determine if the optical characteristics meet the optical characteristic threshold within a second latency duration. The pump module 706 may refrain from modulating the infusion pump when the optical characteristics exceed the optical characteristic threshold within the second latency duration.

Several options for the method 1100 follow. For instance, the method 1100 may include recording the patient-specific dosage of the imaging substance. In an example, the patient-specific dosage may include the first specified volume and the second specified volume of the imaging substance. The method 1100 may include modulating the infusion pump to infuse the patient-specific dosage. In an example, the physiological characteristic identification module 704 may determine the plasma volume of the patient using the recorded optical characteristics and the patient-specific imaging substance dosage.

FIG. 12 illustrates another cross-sectional view of the cuvette of FIG. 2 at the line 3-3, according to an example of the present subject matter. In an example, the cuvette 128 may couple with the blood circuit 120, for instance coupling with the withdrawal line 104, In another example, a profile (e.g., one or more of cross-section, shape, size, dimensions, contour, radius, perimeter, circumference, diameter, outline, boundary, configuration, pattern, arrangement, thickness, or the like) of the cuvette 128 (shown in FIG. 2) corresponds to a profile of one or more of the withdrawal line 104 or the infusion line 106. In yet another example, the withdrawal line 104 may have a withdrawal line profile 1200 and the withdrawal lumen 300 may have a withdrawal lumen profile 1202. The cuvette 128 may have a socket profile 1204, and the cuvette lumen 200 of the cuvette 128 may have a cuvette lumen profile 1206.

In an example, the socket profile 1204 of the cuvette 128 may correspond to the withdrawal line profile 1200. For instance, an outer diameter of the withdrawal line 104 (shown in FIG. 12) may correspond with an inner diameter of the socket 206. In another example, the withdrawal lumen profile 1202 may correspond with the cuvette lumen profile 1206. In yet another example, the withdrawal lumen profile 1202 may correspond with the cuvette lumen profile to align the cuvette lumen 200 with the withdrawal lumen 300. Accordingly, the corresponding profiles of the cuvette and one or more of the lines 104, 106 minimizes discontinuities at an interface between the cuvette 128 and one or more of the lines 104, 106. Thus, disturbance in flow within the cuvette 128 and the lines 104, 106 may be minimized, for instance to minimize clotting the blood circuit 120 (including the cuvette 128). For example, the cuvette 128 may minimize disturbances in flow through the cuvette 128, such as by reducing one or more of eddies or shear forces in the flow of fluid through the cuvette 128 (and the blood circuit 120). For instance, clotting of blood in the cuvette 128 may be minimized because shear forces induced by the cuvette on the blood are reduced because of the correspondence in profiles between the cuvette 128 and one or more of the lines 104, 106.

In yet another example, because the profile of the cuvette 128 corresponds with the profile of one or more of the withdrawal line 104 or the infusion line 106, the system 100 may correct for ambient light received by the photoreceiver, for instance because the cuvette 128 refrains from blocking ambient light (e.g., due to the profile correspondence with one or more of the lines 104, 106). For instance, the optical characteristics of the fluid may include ambient light received by a photoreceiver, such as the photoreceiver 602 (shown in FIG. 6). The calibration module 7097 may compensate for ambient light, for example while the sampling module 702 records the optical characteristics (or determinations based on the optical characteristics).

For instance, the sensor interface module 700 may modulate a photoemitter, such as the photoemitter 600 (shown in FIG. 6) to selectively generate light. The sampling module 702 may record measured optical characteristics, such as optical characteristics corresponding to ambient light received by the photoreceiver. In an example, the sensor interface refrains from generating light while the sampling module records optical characteristics. Accordingly, the calibration module 707 may determine an ambient light correction corresponding to the received ambient light. The calibration module 707 may apply the ambient correction to recorded optical characteristics (e.g., optical characteristics corresponding to an imaging substance, hematocrit, or the like). Thus, the calibration module 707 may provide an ambient corrected optical characteristic. Accordingly, the system 100 may compensate for ambient light received by the optical sensor 130 while measuring optical characteristics of fluid in the blood circuit 120 (shown in FIG. 1). The sampling module may record the ambient light corrected optical characteristic.

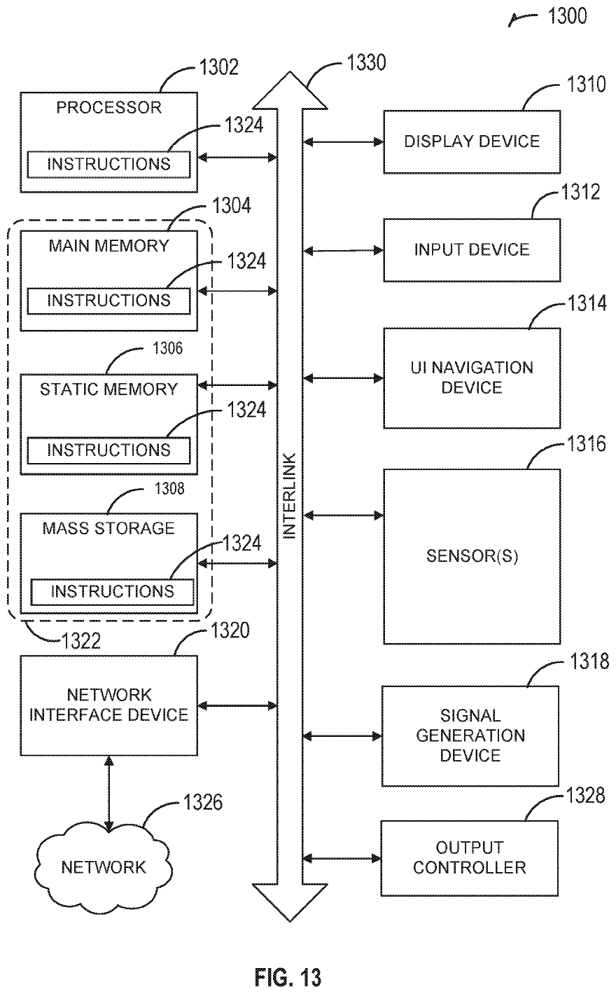

FIG. 13 illustrates a block diagram of an example machine 1300 upon which any one or more of the techniques (e.g., methodologies) discussed herein may perform. Examples, as described herein, may include, or may operate by, logic or a number of components, or mechanisms in the machine 1300. Circuitry (e.g., processing circuitry) is a collection of circuits implemented in tangible entities of the machine 1300 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1300 follow.

In alternative embodiments, the machine 1300 may operate as a standalone device or may be connected (e.g., networked) to other machines. In a networked deployment, the machine 1300 may operate in the capacity of a server machine, a client machine, or both in server-client network environments. In an example, the machine 1300 may act as a peer machine in peer-to-peer (P2P) (or other distributed) network environment. The machine 1300 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 1300 may include a hardware processor 1302 (e.g., a central processing unit (CPU), a graphics processing unit (GPU), a hardware processor core, or any combination thereof), a main memory 1304, a static memory (e.g., memory or storage for firmware, microcode, a basic-input-output (BIOS), unified extensible firmware interface (UEFI), etc.) 1306, and mass storage 1308 (e.g., hard drive, tape drive, flash storage, or other block devices) some or all of which may communicate with each other via an interlink (e.g., bus) 1330. The machine 1300 may further include a display unit 1310, an alphanumeric input device 1312 (e.g., a keyboard), and a user interface (UI) navigation device 1314 (e.g., a mouse). In an example, the display unit 1310, input device 1312 and UI navigation device 1314 may be a touch screen display. The machine 1300 may additionally include a storage device (e.g., drive unit) 1308, a signal generation device 1318 (e.g., a speaker), a network interface device 1320, and one or more sensors 1316, such as a global positioning system (GPS) sensor, compass, accelerometer, or other sensor. The machine 1300 may, include an output controller 1328, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc. Registers of the processor 1302, the main memory 1304, the static memory 1306, or the mass storage 1308 may be, or include, a machine readable medium 1322 on which is stored one or more sets of data structures or instructions 1324 (e.g., software) embodying or utilized by any one or more of the techniques or functions described herein. The instructions 1324 may also reside, completely or at least partially, within any of registers of the processor 1302, the main memory 1304, the static memory 1306, or the mass storage 1308 during execution thereof by the machine 1300. In an example, one or any combination of the hardware processor 1302, the main memory 1304, the static memory 1306, or the mass storage 1308 may constitute the machine readable media 1322. While the machine readable medium 1322 is illustrated as a single medium, the term “machine readable medium” may include a single medium or multiple media (e.g., a centralized or distributed database, and/or associated caches and servers) configured to store the one or more instructions 1324.

The term “machine readable medium” may include any medium that is capable of storing, encoding, or carrying instructions for execution by the machine 1300 and that cause the machine 1300 to perform any one or more of the techniques of the present disclosure, or that is capable of storing, encoding or carrying data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1324 may be further transmitted or received over a communications network 1326 using a transmission medium via the network interface device 1320 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1320 may include one or more physical jacks (e.g., Ethernet, coaxial, or phone jacks) or one or more antennas to connect to the communications network 1326. In an example, the network interface device 1320 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1300, and includes digital or analog communications signals or other intangible medium to facilitate communication of such software. A transmission medium is a machine readable medium.

### VARIOUS NOTES & EXAMPLES

Example 1 is a blood filtration system, comprising: an extracorporeal blood circuit configured to transmit a fluid within one or more lumens, the lumens configured for communication with vasculature including reception of fluid from a patient and infusion of the fluid to the patient; an optical sensor configured to couple with the extracorporeal blood circuit, wherein the optical sensor is configured to measure one or more optical characteristics of the fluid in the extracorporeal blood circuit, the one or more optical characteristics including a first optical characteristic corresponding to a concentration of an imaging substance in the fluid within the blood circuit; and a controller in communication with the optical sensor; wherein the controller includes: a sampling module configured to record the one or more optical characteristics; and a physiological characteristic identification module configured to determine a plasma volume of the patient with the recorded optical characteristics of the imaging substance.

In Example 2, the subject matter of Example 1 optionally includes wherein: the physiological characteristic identification module is configured to determine the concentration of the imaging substance in the fluid within the blood circuit based on the one or more optical characteristics; and the physiological characteristic identification module is configured to determine the plasma volume using the concentration of the imaging substance and an infused volume of the imaging substance.

In Example 3, the subject matter of Example 2 optionally includes wherein: the physiological characteristic identification module is configured to determine an extrapolated concentration of the imaging substance, including: the physiological characteristic identification module fits the recorded optical characteristics to a decay function; and the physiological characteristic identification module extrapolates along the decay function to determine the extrapolated concentration of the imaging substance.

In Example 4, the subject matter of Example 3 optionally includes wherein the physiological characteristic identification module determination of the plasma volume of the patient includes the physiological characteristic identification module dividing the infusion volume of the imaging substance by the extrapolated concentration of the imaging substance.

In Example 5, the subject matter of any one or more of Examples 3-4 optionally include wherein the extrapolated concentration is included in a mixing period of the decay function as the imaging substance mixes with fluid in intravascular space of the patient.

In Example 6, the subject matter of any one or more of Examples 3-5 optionally include wherein: the sampling module is configured to record the extrapolated concentration.

In Example 7, the subject matter of any one or more of Examples 1-6 optionally include an infusion pump configured to infuse a specified volume of the imaging substance, wherein the controller includes a pump module configured to modulate the infusion pump to infuse the specified volume of the imaging substance.

In Example 8, the subject matter of Example 7 optionally includes wherein the recording module is configured to record the optical characteristics for a specified time period when the pump module modulates the infusion pump.

In Example 9, the subject matter of any one or more of Examples 7-8 optionally include wherein: the controller includes a calibration module configured to compare the measured one or more optical characteristics from the optical sensor to an optical characteristic threshold; and the calibration module measures a latency duration between when the pump module modulates the infusion pump and when the measured one or more optical characteristics exceed the optical characteristic threshold.

In Example 10, the subject matter of Example 9 optionally includes wherein: the calibration module is configured to compare the latency duration to a latency threshold; and the sampling module is configured to refrain from recording the optical characteristics for a specified time period when the latency duration exceeds the latency threshold.

In Example 11, the subject matter of any one or more of Examples 7-10 optionally include wherein: the controller includes a calibration module configured to compare the measured one or more optical characteristics from the optical sensor to an optical characteristic threshold; the calibration module is configured to determine if the one or more optical characteristics exceed the optical characteristic threshold within a first latency duration; the specified volume of the imaging substance infused by the infusion pump is a first specified volume of the imaging substance; and the pump module modulates the infusion pump to infuse a second specified volume of the imaging substance when the one or more optical characteristics refrain from exceeding the optical characteristic threshold within the first latency duration.

In Example 12, the subject matter of Example 11 optionally includes wherein: the calibration module is configured to determine if the one or more optical characteristics exceed the optical characteristic threshold within a second latency duration; and the pump module refrains from modulating the infusion pump when the one or more optical characteristics exceed the optical characteristic threshold within the second latency duration.

In Example 13, the subject matter of Example 12 optionally includes wherein: the sampling module is configured to record a patient-specific imaging substance dosage, the patient-specific imaging substance dosage including the first specified volume and the second specified volume of the imaging substance; the pump module is configured to modulate the infusion pump to infuse the patient-specific imaging substance dosage; and the physiological characteristic identification module is configured to determine the plasma volume using the recorded optical characteristics and the patient-specific imaging substance dosage.

In Example 14, the subject matter of Example 13 optionally includes wherein the physiological characteristic identification module is configured to repeatedly determine the plasma volume of the patient using the recorded optical characteristics and the patient-specific imaging substance dosage.

In Example 15, the subject matter of any one or more of Examples 11-14 optionally include wherein: the calibration module is configured to determine if the one or more optical characteristics exceed the optical characteristic threshold with a second latency duration; and the pump module modulates the infusion pump to infuse the imaging substance when the one or more optical characteristics refrain from exceeding the optical characteristic threshold with the second latency duration.

In Example 16, the subject matter of any one or more of Examples 1-15 optionally include wherein the physiological characteristic identification module is configured to repeatedly determine the plasma volume of the patient.

In Example 17, the subject matter of any one or more of Examples 1-16 optionally include wherein: the controller includes a pump module configured to adjust a speed of one or more of a blood pump or a filtration pump based on the plasma volume determined by the physiological characteristic identification module.

In Example 18, the subject matter of Example 17 optionally includes wherein the pump module is configured to adjust the speed of one or more of a blood pump or a filtration pump based on the plasma volume determined by the physiological characteristic identification module, wherein: the pump module is configured to modulate the blood pump to adjust a blood flow rate through a filter of the blood filtration system; and the pump module is configured to modulate the filtration pump to adjust an extraction rate of filtrate fluid from the filter.

In Example 19, the subject matter of any one or more of Examples 17-18 optionally include a hematocrit sensor configured to measure hematocrit value of the patient, wherein the pump module is configured to adjust a filtration fraction based on one or more of the plasma volume determined by the physiological characteristic identification module and the hematocrit value of the patient.

In Example 20, the subject matter of Example 19 optionally includes wherein: the one or more optical characteristics of the fluid in the extracorporeal blood circuit includes a second optical characteristic corresponding to a concentration of one or more plasma constituents in the fluid within the blood circuit; the optical sensor is configured to measure the second optical characteristic; and the physiological characteristic identification module is configured to determine the hematocrit value of the patient with the recorded optical characteristics.

In Example 21, the subject matter of any one or more of Examples 17-20 optionally include wherein: the physiological characteristic identification module is configured to compare the determined plasma volume to a plasma volume threshold; and the pump module is configured to adjust the speed of one or more of blood pump or the filtration pump when the determined plasma volume exceeds the plasma volume threshold.

In Example 22, the subject matter of any one or more of Examples 1-21 optionally include a hematocrit sensor configured to measure hematocrit value of the patient, wherein: a sensor interface module included in the controller, the sensor interface module configured to receive the measured hematocrit value from the hematocrit sensor.

In Example 23, the subject matter of Example 22 optionally includes wherein: the physiological characteristic identification module is configured to determine a red blood cell volume of the patient using the determined plasma volume and the measured hematocrit value.

In Example 24, the subject matter of any one or more of Examples 22-23 optionally include wherein: the one or more optical characteristics of the fluid in the extracorporeal blood circuit includes a second optical characteristic corresponding to a concentration of one or more plasma constituents in the fluid within the blood circuit; the hematocrit sensor is included in the optical sensor, and the optical sensor is configured to measure the second optical characteristic the one or more plasma constituents; and the physiological characteristic identification module is configured to determine the hematocrit value of the patient with the recorded optical characteristics.

In Example 25, the subject matter of any one or more of Examples 1-24 optionally include wherein the concentration of the imaging substance in the fluid within the blood circuit corresponds to the plasma volume of the patient.

In Example 26, the subject matter of any one or more of Examples 1-25 optionally include wherein the concentration of the imaging substance in the fluid within the blood circuit corresponds to a hematocrit value of the patient.

In Example 27, the subject matter of any one or more of Examples 1-26 optionally include wherein the optical sensor includes: a photoemitter configured to generate light at a specified optical characteristic, wherein the photoemitter is configured to transmit the light through a component of the blood circuit; and a photoreceiver configured to measure the optical characteristics of the fluid in the extracorporeal blood circuit.

In Example 28, the subject matter of Example 27 optionally includes wherein the physiological characteristic identification module is configured to determine the concentration of the imaging substance in the fluid within the blood circuit.

In Example 29, the subject matter of Example 28 optionally includes wherein the physiological characteristic identification module is configured to compare the measured optical characteristics of the fluid in the blood circuit to the specified optical characteristic to determine to concentration of the imaging substance in the fluid in the blood circuit.

In Example 30, the subject matter of any one or more of Examples 27-29 optionally include wherein: the one or more optical characteristics of the fluid in the blood circuit includes a third optical characteristic corresponding to ambient light received by the photoreceiver; and the controller includes a calibration module configured to compensate for the third optical characteristic while the sampling module records the optical characteristics.

In Example 31, the subject matter of Example 30 optionally includes wherein: the controller includes a sensor interface configured to modulate the photoemitter to selectively generate the light at the specified optical characteristic; the sampling module is configured to record the third optical characteristic when the sensor interface refrains from generating the light; the calibration module is configured to determine an ambient correction corresponding to the third optical characteristic; the calibration module is configured to apply the ambient correction to the recorded optical characteristics to provide ambient corrected optical characteristics of the fluid in the extracorporeal blood circuit; and the sampling module is configured to record the ambient corrected optical characteristics.

In Example 32, the subject matter of any one or more of Examples 30-31 optionally include a blood flow sensor configured to measure blood flow rate within at least one of the one or more lumens.

In Example 33, the subject matter of any one or more of Examples 1-32 optionally include wherein the extracorporeal blood circuit includes a filter, and the filter is configured to remove one or more plasma constituents from the fluid in the extracorporeal blood circuit.

Example 34 is a method for administering a patient-specific dosage of an imaging substance, the method comprising: modulating an infusion pump to infuse a first specified volume of the imaging substance into vasculature of the patient; monitoring one or more optical characteristics of fluid in a blood circuit, the blood circuit in communication with the vasculature of the patient;

comparing the optical characteristics of the fluid in the blood circuit to an optical characteristic threshold; and modulating the infusion pump to infuse a second specified volume of the imaging substance when the one or more optical characteristics refrain from exceeding the optical characteristic threshold.

In Example 35, the subject matter of Example 34 optionally includes wherein the second specified volume is infused when the one or more optical characteristics refrain from exceeding the optical characteristic threshold within a latency duration.

In Example 36, the subject matter of any one or more of Examples 34-35 optionally include recording the patient-specific dosage of the imaging substance, the patient-specific dosage including the first specified volume and the second specified volume of the imaging substance.

In Example 37, the subject matter of any one or more of Examples 35-36 optionally include modulating the infusion pump to infuse the patient-specific dosage.

Example 38 is a method for removing one or more plasma constituents from blood of a patient, comprising: setting an extraction rate of a filtration pump, the filtration pump configured to extract a filtrate fluid from a filter, wherein: the filter is configured to receive blood and remove the one or more plasma constituents and provide a filtrate fluid including the one or more plasma constituents; monitoring a plasma volume of a patient; comparing the plasma volume of the patient to a target plasma volume for the patient to determine a difference between the plasma volume of the patient and target plasma volume for the patient; and modulating the filtration pump to vary the extraction rate based on the difference between the plasma volume of the patient and the target plasma volume of the patient.

In Example 39, the subject matter of Example 38 optionally includes wherein modulating the filtration pump includes varying the extraction rate in proportion to the difference between the plasma volume of the patient and the target plasma volume of the patient.

In Example 40, the subject matter of any one or more of Examples 38-39 optionally include modulating the filtration pump to set the extraction rate to zero when the plasma volume of the patient exceeds the target plasma volume.

In Example 41, the subject matter of Example 40 optionally includes modulating a blood pump to set a blood flow rate to zero after a specified time duration.

Example 42 is a blood filtration system; comprising: an extracorporeal blood circuit configured to transmit a fluid within one or more lumens, the lumens configured for communication with vasculature including reception of fluid from the patient and infusion of the fluid to a patient; an optical sensor configured to couple with the extracorporeal blood circuit, wherein the optical sensor is configured to measure one or more optical characteristics of the fluid in the extracorporeal blood circuit, the one or more optical characteristics including a first optical characteristic corresponding to a concentration of an imaging substance in the fluid within the blood circuit; and a controller in communication with the optical sensor, wherein the controller includes: a sampling module configured to record the one or more optical characteristics; and a physiological characteristic identification module configured to determine a plasma volume of the patient with the recorded optical characteristics of the imaging substance, wherein: the physiological characteristic identification module is configured to determine the concentration of the imaging substance in the fluid within the blood circuit based on the one or more optical characteristics; and the physiological characteristic identification module is configured to determine the plasma volume using the concentration of the imaging substance and an infused volume of the imaging substance.

In Example 43, the subject matter of Example 42 optionally includes wherein: the physiological characteristic identification module is configured to determine an extrapolated concentration of the imaging substance, including: the physiological characteristic identification module fits the recorded optical characteristics to a decay function; and the physiological characteristic identification module extrapolates along the decay function to determine the extrapolated concentration of the imaging substance.

In Example 44, the subject matter of Example 43 optionally includes wherein the physiological characteristic identification module determination of the plasma volume of the patient includes the physiological characteristic identification module dividing the infusion volume of the imaging substance by the extrapolated concentration of the imaging substance.

In Example 45, the subject matter of any one or more of Examples 43-44 optionally include wherein the extrapolated concentration is included in a mixing period of the decay function as the imaging substance mixes with fluid in intravascular space of the patient.

In Example 46, the subject matter of any one or more of Examples 43-45 optionally include wherein: the sampling module is configured to record the extrapolated concentration.

In Example 47, the subject matter of any one or more of Examples 42-46 optionally include an infusion pump configured to infuse a specified volume of the imaging substance, wherein the controller includes a pump module configured to modulate the infusion pump to infuse the specified volume of the imaging substance.

In Example 48, the subject matter of any one or more of Examples 42-47 optionally include wherein the physiological characteristic identification module is configured to determine a hematocrit value of the patient.

In Example 49, the subject matter of any one or more of Examples 42-48 optionally include wherein the physiological characteristic identification module is configured to determine a plasma refill rate of the patient.

In Example 50, the subject matter of any one or more of Examples 42-49 optionally include wherein the physiological characteristic identification module is configured to determine a cardiac output of the patient.

In Example 51, the subject matter of any one or more of Examples 42-50 optionally include wherein the controller includes a pump module configured to modulate one or more of a blood pump or an infusion pump, wherein the controller modulates one or more of the blood pump or the infusion pump based on the determined plasma volume of the patient.

In Example 52, the subject matter of any one or more of Examples 42-51 optionally include a cuvette included in the blood circuit, wherein the optical sensor is configured to couple with the cuvette.

In Example 53, the subject matter of Example 52 optionally includes wherein the optical sensor is configured to transmit light through an optical window of the cuvette.

In Example 54, the subject matter of any one or more of Examples 42-53 optionally include wherein: the one or more lumens are a withdrawal lumen or an infusion lumen; the extracorporeal blood circuit includes a cuvette having a cuvette lumen in communication with the withdrawal lumen or the infusion lumen; the withdrawal lumen has a withdrawal lumen profile; the infusion lumen has an infusion lumen profile; the cuvette lumen has a cuvette lumen profile; and the cuvette lumen profile corresponds with one or more of the withdrawal lumen profile or the infusion lumen profile.

Example 55 may include or use, or may optionally be combined with any portion or combination of any portions of any one or more of Examples 1 through 54 to include or use, subject matter that may include means for performing any one or more of the functions of Examples 1 through 54, or a machine-readable medium including instructions that, when performed by a machine, cause the machine to perform any one or more of the functions of Aspects 1 through 54.

Each of these non-limiting examples may stand on its own, or may be combined in various permutations or combinations with one or more of the other examples. The above description includes references to the accompanying drawings, which form a part of the detailed description. The drawings show, by, way of illustration; specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is provided to comply with 37 C.F.R. § 1.72(b), to allow the reader to quickly ascertain the nature of the technical disclosure, it is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments may be combined with each other in various combinations or permutations. The scope of the invention should be determined with reference to the appended claims, along with the full scope of equivalents to which such claims are entitled.

1. A blood filtration system, comprising:
a blood circuit configured to transmit a fluid within one or more lumens, the lumens configured for communication with vasculature including reception of fluid from a patient and infusion of the fluid to the patient;
an optical sensor configured to couple with the extracorporeal blood circuit, wherein the optical sensor is configured to measure one or more optical characteristics of the fluid in the extracorporeal blood circuit, the one or more optical characteristics including a first optical characteristic corresponding to a concentration of an imaging substance in the fluid within the blood circuit; and
a controller in communication with the optical sensor, wherein the controller includes:
a sampling module configured to record the one or more optical characteristics; and
a physiological characteristic identification module configured to determine a plasma volume of the patient with the recorded optical characteristics of the imaging substance.

2. The blood filtration system of claim 1, wherein:
the physiological characteristic identification module is configured to determine the concentration of the imaging substance in the fluid within the blood circuit based on the one or more optical characteristics; and
the physiological characteristic identification module is configured to determine the plasma volume using the concentration of the imaging substance and an infused volume of the imaging substance.

3. The blood filtration system of claim 2, wherein:
the physiological characteristic identification module is configured to determine an extrapolated concentration of the imaging substance, including:
the physiological characteristic identification module fits the recorded optical characteristics to a decay function; and
the physiological characteristic identification module extrapolates along the decay function to determine the extrapolated concentration of the imaging sub stance.

4. The blood filtration system of claim 3, wherein the physiological characteristic identification module determination of the plasma volume of the patient includes the physiological characteristic identification module dividing the infusion volume of the imaging substance by the extrapolated concentration of the imaging substance.

5. The blood filtration system of claim 3, wherein the extrapolated concentration is included in a mixing period of the decay function as the imaging substance mixes with fluid in intravascular space of the patient.

6. The blood filtration system of claim 3, wherein:
the sampling module is configured to record the extrapolated concentration.

7. The blood filtration system of claim 1, further comprising an infusion pump configured to infuse a specified volume of the imaging substance, wherein the controller includes a pump module configured to modulate the infusion pump to infuse the specified volume of the imaging substance.

8. The blood filtration system of claim 7, wherein the recording module is configured to record the optical characteristics for a specified time period when the pump module modulates the infusion pump.

9. The blood filtration system of claim 7, wherein:
the controller includes a calibration module configured to compare the measured one or more optical characteristics from the optical sensor to an optical characteristic threshold; and
the calibration module measures a latency duration between when the pump module modulates the infusion pump and when the measured one or more optical characteristics exceed the optical characteristic threshold.

10. The blood filtration system of claim 9, wherein:
the calibration module is configured to compare the latency duration to a latency threshold; and
the sampling module is configured to refrain from recording the optical characteristics for a specified time period when the latency duration exceeds the latency threshold.

11. The blood filtration system of claim 7, wherein:
the controller includes a calibration module configured to compare the measured one or more optical characteristics from the optical sensor to an optical characteristic threshold;
the calibration module is configured to determine if the one or more optical characteristics exceed the optical characteristic threshold within a first latency duration;
the specified volume of the imaging substance infused by the infusion pump is a first specified volume of the imaging substance; and
the pump module modulates the infusion pump to infuse a second specified volume of the imaging substance when the one or more optical characteristics refrain from exceeding the optical characteristic threshold within the first latency duration.

12. The blood filtration system of claim 11, wherein:
the calibration module is configured to determine if the one or more optical characteristics exceed the optical characteristic threshold within a second latency duration; and
the pump module refrains from modulating the infusion pump when the one or more optical characteristics exceed the optical characteristic threshold within the second latency duration.

13. The blood filtration system of claim 12, wherein:
the sampling module is configured to record a patient-specific imaging substance dosage, the patient-specific imaging substance dosage including the first specified volume and the second specified volume of the imaging substance;
the pump module is configured to modulate the infusion pump to infuse the patient-specific imaging substance dosage; and
the physiological characteristic identification module is configured to determine the plasma volume using the recorded optical characteristics and the patient-specific imaging substance dosage.

14. The blood filtration system of claim 13, wherein the physiological characteristic identification module is configured to repeatedly determine the plasma volume of the patient using the recorded optical characteristics and the patient-specific imaging substance dosage.

15. The blood filtration system of claim 11, wherein:
the calibration module is configured to determine if the one or more optical characteristics exceed the optical characteristic threshold with a second latency duration; and
the pump module modulates the infusion pump to infuse the imaging substance when the one or more optical characteristics refrain from exceeding the optical characteristic threshold with the second latency duration.

16. The blood filtration system of claim 1, wherein the physiological characteristic identification module is configured to repeatedly determine the plasma volume of the patient.

17. The blood filtration system of claim 1, wherein:
the controller includes a pump module configured to adjust a speed of one or more of a blood pump or a filtration pump based on the plasma volume determined by the physiological characteristic identification module.

18. The blood filtration system of claim 17, wherein the pump module is configured to adjust the speed of one or more of a blood pump or a filtration pump based on the plasma volume determined by the physiological characteristic identification module, wherein:
the pump module is configured to modulate the blood pump to adjust a blood flow rate through a filter of the blood filtration system; and
the pump module is configured to modulate the filtration pump to adjust an extraction rate of filtrate fluid from the filter.

19. The blood filtration system of claim 17, further comprising a hematocrit sensor configured to measure hematocrit value of the patient, wherein the pump module is configured to adjust a filtration fraction based on one or more of the plasma volume determined by the physiological characteristic identification module and the hematocrit value of the patient.

20. The blood filtration system of claim 19, wherein:
the one or more optical characteristics of the fluid in the extracorporeal blood circuit includes a second optical characteristic corresponding to a concentration of one or more plasma constituents in the fluid within the blood circuit;
the optical sensor is configured to measure the second optical characteristic; and
the physiological characteristic identification module is configured to determine the hematocrit value of the patient with the recorded optical characteristics.

21. The blood filtration system of claim 17, wherein:
the physiological characteristic identification module is configured to compare the determined plasma volume to a plasma volume threshold; and
the pump module is configured to adjust the speed of one or more of blood pump or the filtration pump when the determined plasma volume exceeds the plasma volume threshold.

22. The blood filtration system of claim 1, further comprising:
a hematocrit sensor configured to measure hematocrit value of the patient, wherein:
a sensor interface module included in the controller, the sensor interface module configured to receive the measured hematocrit value from the hematocrit sensor.

23. The blood filtration system of claim 22, wherein:
the physiological characteristic identification module is configured to determine a red blood cell volume of the patient using the determined plasma volume and the measured hematocrit value.

24. The blood filtration system of claim 22, wherein:
the one or more optical characteristics of the fluid in the extracorporeal blood circuit includes a second optical characteristic corresponding to a concentration of one or more plasma constituents in the fluid within the blood circuit;
the hematocrit sensor is included in the optical sensor, and the optical sensor is configured to measure the second optical characteristic the one or more plasma constituents; and
the physiological characteristic identification module is configured to determine the hematocrit value of the patient with the recorded optical characteristics.

25. The blood filtration system of claim 1, wherein the concentration of the imaging substance in the fluid within the blood circuit corresponds to the plasma volume of the patient.

26. The blood filtration system of claim 1, wherein the concentration of the imaging substance in the fluid within the blood circuit corresponds to a hematocrit value of the patient.

27. The blood filtration system of claim 1, wherein the optical sensor includes:
a photoemitter configured to generate light at a specified optical characteristic, wherein the photoemitter is configured to transmit the light through a component of the blood circuit; and
a photoreceiver configured to measure the optical characteristics of the fluid in the extracorporeal blood circuit.

28. The blood filtration system of claim 27, wherein the physiological characteristic identification module is configured to determine the concentration of the imaging substance in the fluid within the blood circuit.

29. The blood filtration system of claim 28, wherein the physiological characteristic identification module is configured to compare the measured optical characteristics of the fluid in the blood circuit to the specified optical characteristic to determine to concentration of the imaging substance in the fluid in the blood circuit.

30. The blood filtration system of claim 27, wherein:
the one or more optical characteristics of the fluid in the blood circuit includes a third optical characteristic corresponding to ambient light received by the photoreceiver; and
the controller includes a calibration module configured to compensate for the third optical characteristic while the sampling module records the optical characteristics.

31. The blood filtration system of claim 30, wherein:
the controller includes a sensor interface configured to modulate the photoemitter to selectively generate the light at the specified optical characteristic;
the sampling module is configured to record the third optical characteristic when the sensor interface refrains from generating the light;
the calibration module is configured to determine an ambient correction corresponding to the third optical characteristic;
the calibration module is configured to apply the ambient correction to the recorded optical characteristics to provide ambient corrected optical characteristics of the fluid in the extracorporeal blood circuit; and
the sampling module is configured to record the ambient corrected optical characteristics.

32. The blood filtration system of claim 30, further comprising a blood flow sensor configured to measure blood flow rate within at least one of the one or more lumens.

33. The blood filtration system of claim 1, wherein the extracorporeal blood circuit includes a filter, and the filter is configured to remove one or more plasma constituents from the fluid in the extracorporeal blood circuit.

34-54. (canceled)
